(19) United States
(12) Patent Application Publication
(10) Pub. No.: US 2025/0121185 A1
(43) Pub. Date: Apr. 17, 2025

(54) ALTERNATING ELECTRODES BETWEEN MEASUREMENT AND INTERVENTION MODES TO ADDRESS HYPEREXCITABILITY

(71) Applicant: Cionic, Inc., San Francisco, CA (US)

(72) Inventor: Jeremiah Robison, San Francisco, CA (US)

(21) Appl. No.: 18/913,824

(22) Filed: Oct. 11, 2024

Related U.S. Application Data
(60) Provisional application No. 63/590,160, filed on Oct. 13, 2023.

(51) Int. Cl. A61N 1/36; A61N 1/04

(52) U.S. Cl. CPC A61N 1/36031; A61N 1/0452; A61N 1/0472; A61N 1/36003

(57) ABSTRACT

A mobility augmentation system configures electrodes of a wearable stimulation array to operate in measurement and intervention modes to detect hyperexcitability of one or more muscles of a user. In the measurement mode, electrodes of the array measure electromyography (EMG) signals from one or more muscles of a user. If the measured EMG signal indicates muscle hyperexcitability, the set of electrodes is configured to operate in the intervention mode and applies an intervention signal to the muscle(s). After the intervention signal is applied, the set of electrodes are reconfigured to return to the measurement mode and a second EMG signal is measured. In response to determining that the intervention signal did not reduce the hyperexcitability of the muscle(s) by at least a threshold amount, the electrodes are returned to the intervention mode and apply a second intervention signal based on the second EMG signal.

### CROSS-REFERENCE TO RELATED APPLICATIONS

This application claims priority to U.S. Provisional Application No. 63/590,160, filed Oct. 13, 2023, which is incorporated by reference.

### TECHNICAL FIELD

This disclosure relates generally to a mobility augmentation system, and more specifically to configuring electrodes of a wearable stimulation array to operate in measurement and intervention modes to address detected muscle hyperexcitability.

### BACKGROUND

While neurostimulation systems are often used to augment a series of movements by a user, they can also be used to combat undesired motor function activity, such as spastic muscle contractions or other hyperexcitability events. In this context, systems can apply electromyography (EMG) signals to measure muscle hyperexcitability and deliver appropriate stimulation to reduce or eliminate such events. Existing systems, however, designate different sets of electrodes to carry out each of these tasks. For example, a first set of electrodes might be used to capture EMG data for detecting and quantifying hyperexcitability events, while a second set of electrodes are used to deliver intervention signals to reduce the detected hyperexcitability. This separation hampers the ability of these systems to operate an efficient feedback loop between real-time assessment of the user's physical state and the responsive application of intervention signals. Such a disconnect between measurement and responsive interventions could lead to inaccuracies in the applied intervention or delay the provision of necessary stimulation.

### SUMMARY

The wearable stimulation array and mobility augmentation system described herein enables dynamic movement stimulation and intervention. In one embodiment, the wearable stimulation array includes a set of configurable electrodes each configured to contact a different portion of a surface of a body of a user when the wearable stimulation array is worn by the user. The wearable stimulation array includes an electrode multiplexer (MUX) that enables the dynamic reconfiguration of the array's electrodes to operate in one or more of various roles (e.g., anode, cathode, or disconnected) and in different modes (e.g., measurement mode or intervention mode) and to apply different types of signals (e.g., afferent signals or efferent signals). The mobility augmentation system applies a movement model (e.g., a machine learned model) to measurements taken by sensors at the wearable stimulation array. The model can determine an actuation instruction for each of various movements. For example, the model can determine electrical stimulation for each movement in a gait cycle, enabling an electrical signal to flow from one set of electrodes and to another set of electrodes, where the electrode configuration can change for each movement.

The wearable stimulation array is further configured to operate in a measurement mode and an intervention mode. In the measurement mode, the set of electrodes measure electromyography (EMG) signals from one or more muscles of a user. Responsive to determining that a measured EMG signal is representative of muscle hyperexcitability (e.g., based on a comparison between one or more parameters of the EMG signal and at least one hyperexcitability threshold), the set of electrodes is configured to operate in an intervention mode such that the set of electrodes applies an intervention signal to the one or more muscles.

After the intervention signal is applied, the set of electrodes are reconfigured to return to the measurement mode, and a second EMG signal is measured from the one or more muscles. In response to determining that the intervention signal did not reduce the hyperexcitability of the one or more muscles by at least a threshold amount, the array configures the set of electrodes to operate in the intervention mode such that the set of electrodes applies a second intervention signal to the one or more muscles based at least in part on the second EMG signal. Modules of the array may continue to evaluate EMG data during measurement modes and adjust and apply intervention signals during intervention modes until the muscle hyperexcitability is reduced by at least a threshold amount. In this way, the mobility augmentation system of the array operates a feedback loop for optimizing intervention signals applied in response to detected hyperexcitability events.

The wearable stimulation array is further configured to interleave sets of afferent signals and efferent signals to suppress spasticity and stimulate intended movement. Upon initialization of the wearable stimulation array, an initial set of afferent signals are selected and applied by a set of electrodes to establish a neurological baseline of afferent feedback. Responsive to detecting (e.g., based on EMG signal data) spasticity of one or more muscles of the user, the applied set of afferent signals are modified to suppress the detected spasticity, such as by increasing the signal amplitude or frequency or altering the signal pattern. The array also identifies an intended movement of the user, e.g., using EMG sensors capable of detecting specific muscular or neural patterns indicative of an intention to move, and selects a set of efferent signals to facilitate the intended movement. The selected efferent signals are applied in an interleaved fashion with the afferent signals, thereby facilitating the intended movement via functional motor stimulations and maintaining the baseline neuromuscular interaction or suppressing detected spasticity via the afferent pulse train.

### BRIEF DESCRIPTION OF THE DRAWINGS

FIG. 1 is a block diagram of a system environment in which a wearable stimulation array operates, in accordance with at least one embodiment.

FIG. 2 is a block diagram of a wearable stimulation array, in accordance with at least one embodiment.

FIG. 3 is a block diagram of a feedback loop for optimizing stimulation by the wearable stimulation array, in accordance with at least one embodiment.

FIG. 4 depicts electrodes of a wearable stimulation array in contact with a user's shank, in accordance with at least one embodiment.

FIG. 5 is a graphical depiction of EMG measurements captured by electrodes of the wearable stimulation array before and after application of an intervention signal, in accordance with at least one embodiment.

FIG. 6 is a graphical depiction of interleaved afferent and efferent signals applied by a set of electrodes of the wearable stimulation array, in accordance with at least one embodiment.

FIG. 7 is a flowchart illustrating a process for alternating a set of electrodes between measurement and intervention modes to address hyperexcitability, in accordance with at least one embodiment.

FIG. 8 is a flowchart illustrating a process for interleaving afferent and efferent signals applied by a set of electrodes of the wearable stimulation array, in accordance with at least one embodiment.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

### System Architecture

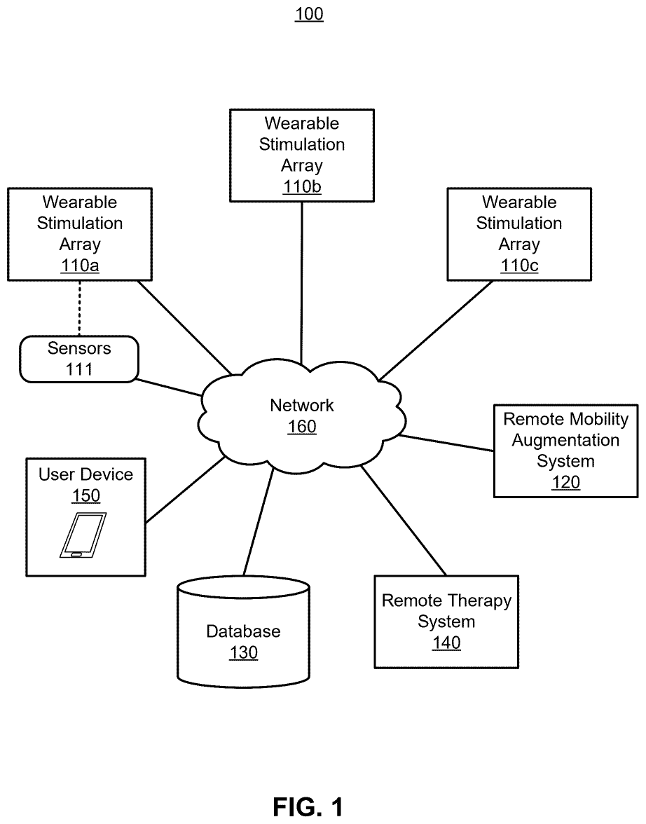

FIG. 1 is a block diagram of a system environment 100 in which a wearable stimulation array operates, in accordance with at least one embodiment. The system environment 100 shown by FIG. 1 includes wearable stimulation arrays 110a-c, sensors 111, a remote mobility augmentation system 120, a database 130, a remote therapy system 140, a user device 150, and a network 160. The system environment 100 may have alternative configurations than shown in FIG. 1, including for example different, fewer, or additional components. For example, the remote therapy system 140 may be omitted from the system environment 100 without compromising the functionality of the wearable stimulation arrays 110a-c.

The system environment 100 enables personalized and dynamic movement augmentation and intervention. Each wearable stimulation array is configured to apply actuation that is optimized based on the user's feedback of the actuation. For example, the wearable stimulation array can calibrate default actuation depending on their effects on the user. The array may iterate through a variety of actuations for a movement and allow the user to provide feedback indicating which of the actuations is preferred for the movement (e.g., the most comfortable or effective). The array may perform this iterative calibration for multiple movements, such as the movements within a gait cycle. The array can iterate through actuations by automatically adjusting, for example, parameters of an electrical signal and which of the electrodes of the array operate as anodes or cathodes. In this way, the array receives user feedback for various actuation of a variety of movements and can maintain scores based on the feedback for each attempted actuation. The highest scoring actuation can replace a default actuation to customize the array to the user. The present wearable stimulation array provides the advantage of actuation adjustment without requiring a user to manually change the electrodes or the placement of the electrodes.

Additionally, the wearable stimulation array can continue to adapt to the user's body during use (e.g., between calibrations), which can change over time due to fatigue, age, injury, or any other stimulus that affects physical movement. For example, the array can determine the efficacy of an actuation by comparing the resultant stimulated movement to a target movement (e.g., a neurotypical movement or a baseline for the user during calibration). The array can use the comparison as feedback to retrain a model that determines the actuation to apply and iterate through various actuations until the resultant stimulated movement is within an acceptable range of the target movement. Yet another benefit of the wearable stimulation array is that the electrical stimulation applied by the array may be applied at the surface of the user's skin, improving the safety and comfort of using the array over invasive stimulation devices with needles that penetrate the user's muscles. Thus, through calibration, automated actuation adjustment, and continued optimization, the wearable stimulation array described herein can provide personalized, non-invasive, and dynamic movement augmentation.

The wearable stimulation arrays 110a-c apply electrical stimulation or other types of actuation to increase the mobility of users or respond to detected hyperexcitability events. The wearable stimulation arrays 110a-c monitor a user's movement to determine current movement (e.g., using IMUs or pressure sensors) or intended movement (e.g., using EMG sensors), and apply actuation based on the monitored movement. The arrays 110a-c may be worn by one or more users. For example, a single user may wear the arrays 110a-c at a forearm, a shank, and a foot, respectively. In another example, a first user may wear the arrays 110a-b, and a second user may wear the array 110c. A wearable stimulation array may include sensors or be communicatively coupled to a sensor. For example, the wearable stimulation array 110a is communicatively coupled to the sensor 111, which may be a camera configured to capture image data of the user's movement for determining an appropriate actuation instruction. Monitoring of EMG sensor data also enables the wearable stimulation arrays 110a-c to detect hyperexcitability events and select and apply intervention signals to reduce muscle hyperexcitability.

The wearable stimulation arrays 110a-c may be worn at various locations on the body of the user to monitor and stimulate movement. For example, the wearable stimulation array 110a may use electromyography to monitor the electrical activity of the user's muscles. From the monitored electrical activity, the array 110a may determine a corresponding actuation for stimulating movement using a movement model (e.g., a machine learning model) trained to identify an actuation from movement data. The terms “movement data” or “movement signal” may refer to data such as kinematic, kinetic, or pressure signals representing a user's physical movement. As referred to herein, “activity data” represents activity of the user's body such as physical movement, electrical muscle activity, heart rate, respiration, any suitable measurement of current movement or intended movement, or combination thereof. Activity data may include movement data. Continuing the earlier example, the array 110a may determine an actuation to apply based on the identified intention. The determined actuation may include instructions to apply electrical stimulation to the various locations on the body of the user. For example, the array 110a at the left shank may be communicatively coupled to the array 110b at the right shank, and the actuation determined by the array 110a may instruct the array 110a to apply a first electrical signal and the array 110b to apply a second electrical signal.

The wearable stimulation arrays 110a-c enable both personalization and optimization of mobility augmentation for their users. The arrays 110a-c may calibrate the actuation to the user's body and continually optimize the actuation as the user wears the arrays. To calibrate the actuation, the arrays 110a-c may first apply a default actuation instruction for respective movements with which they are configured to stimulate. The user may provide feedback for each default actuation instruction (e.g., a measure of approval indicating comfort or efficacy of the actuation), and the arrays 110a-c may use the feedback to modify the actuation until the user feedback indicates that the actuation is satisfactory.

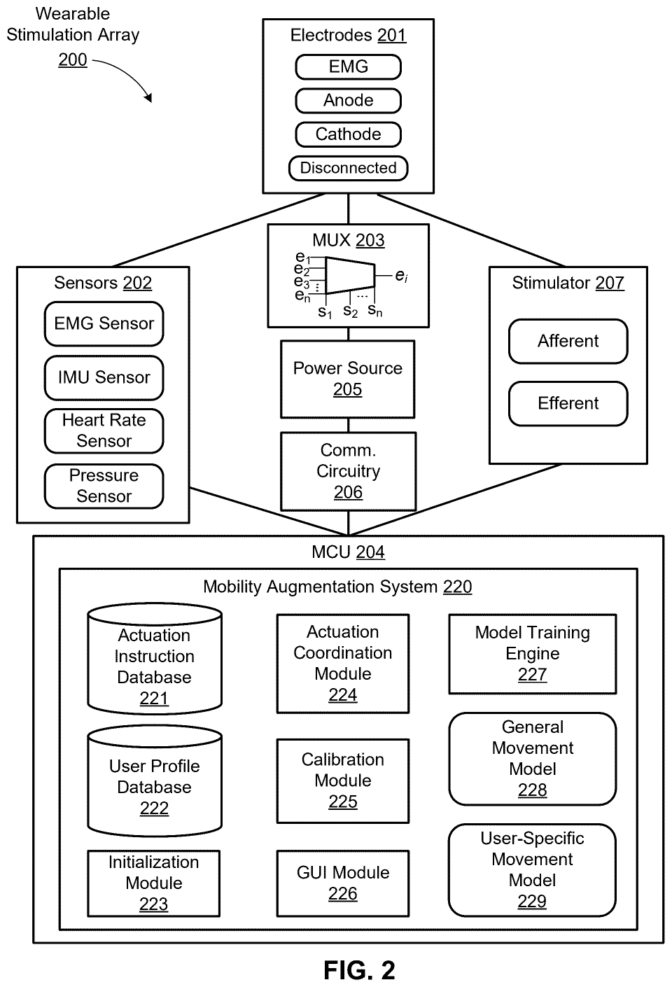

In addition to personalized calibration, another way in which the wearable stimulation arrays 110a-c personalize mobility augmentation is by using movement data collected from a user to train a user-specific machine learning model used to determine the actuation for that user's movements. The arrays 110a-c may optimize mobility augmentation by measuring the success of the actuation in real time (e.g., user feedback) and in response, re-training the machine learning model using the feedback and modifying the subsequently applied actuation. Personalization and optimization will be described in further detail throughout the description of the mobility augmentation system 220 in FIG. 2.

The remote mobility augmentation system 120 receives and processes data from the wearable stimulation arrays 110a-c. The data received from the arrays 110a-c may include movement data, applied actuation, and user feedback. This data may be used to generate new actuation instructions or modify existing actuation instructions. The remote system 120 may use the processed data to provide actuation instructions for the arrays 110a-c to execute. The remote mobility augmentation system 120 may have functionality similar to that of the mobility augmentation system 220 described in FIG. 2. The remote system 120 may be hosted on a server or computing device (e.g., a smartphone) that communicates with the wearable stimulation arrays 110a-c via the network 160.

In some embodiments, the remote mobility augmentation system 120 trains and applies one or more machine learning models configured to determine an actuation instruction based on measured movement data. The remote mobility augmentation system 120 may maintain machine learning models in addition to or alternative to the wearable stimulation arrays 110a-c maintaining the models. In one embodiment, the remote mobility augmentation system 120 trains the models based on movement data collected by the arrays 110a-c. The arrays 110a-c send, via the network 160, movement data to the remote mobility augmentation system 120 and leverage the trained machine learning models to receive, from the remote mobility augmentation system 120, an actuation instruction determined by the one or more models. The remote mobility augmentation system 120 may maintain models that are generalized to movement across a population or customized to a particular user, movement type, any suitable phenotypic trait, or a combination thereof. The training and application of machine learning models used for augmenting mobility is further described in the description of FIG. 2.

The actuation for movement stimulation may be determined by the wearable stimulation arrays 110a-c, the remote mobility augmentation system 120, or manually specified by an operator (e.g., a physical therapist via remote therapy system 140) or the user through an input interface on the user device 150. In some embodiments, the actuation includes electrical stimulation (e.g., a functional electrical stimulation (FES) signal) characterized by a frequency, a pulse duration, duty cycle, and an amplitude (e.g., a value of current in milliamperes). The wearable stimulation array 110a-c may enable various actuation types. Examples of actuation types include manually triggered actuation, amplification, contralateral replay, body-to-body coaching, templated sequencing, and responsive optimization. Examples of actuation by a wearable device may be found in U.S. patents application Ser. Nos. 17/113,058 and 17/113,059, filed on Dec. 6, 2020, which are incorporated herein by reference.

The database 130 stores data related to the operation of the wearable stimulation arrays 110a-c. In some embodiments, the database 130 stores data for training machine learning models of the wearable stimulation arrays 110a-c or the remote mobility augmentation system 120. The data stored in the database 130 may include labeled or unlabeled movement data and labels associated with movements, or templates associated with sequences of muscle firings for given movements. The mobility management system 110 or the mobility augmentation devices 120a and 120b may access the stored data to train machine learning models. The wearable stimulation arrays 110a-c may provide their measured data to the database 130. The provided data may be organized in a data structure including the measured data, biographical information identifying the user and phenotypic traits, and a label identifying an actuation instruction to augment a movement corresponding to the measured data.

In some embodiments, the database 130 stores users' individual movement models in addition to a general model trained on data from across a population. The wearable stimulation arrays may access a model stored in the database 130. For example, a first user who is a stroke survivor may access the movement model of a second user's, who was also a stroke survivor, to begin calibration and optimization from the second user's model rather than a more general movement model that is not yet adapted for stroke survivors.

The remote therapy system 140 enables a third party (e.g., a medical professional or athletic coach) to monitor the user's movement and analyze the information to further augment the user's movement. For example, a physician uses the remote therapy system 140 to monitor their patient's movement and adjust a combination of actuation instructions upon identifying that the patient's movement is not improving under the current actuation instructions. The remote therapy system 140 may be a software module that the third party may execute on a computing device (e.g., a smartphone). In some embodiments, the remote therapy system 140 is a standalone device that may be communicatively coupled to the wearable stimulation arrays 110a-c to manually adjust or generate actuation instructions used to augment the user's movements (e.g., overriding the actuation instructions determined by the wearable stimulation array). The remote therapy system 140 may include an input interface for the third party to specify parameters of an actuation instruction (e.g., the amplitude and frequency of FES signals) and when to apply them.

The remote therapy system 140 may provide actuation strategies to be applied by the mobility augmentation system 220. In some embodiments, a user of the remote therapy system 140 (e.g., a therapist) may specify when to apply stimulation and through which of the wearable stimulation arrays 110a-c to apply stimulation. For example, the therapist may define where, when, and how (e.g., parameters of electrical signals) to stimulate the patient's gait based on a video camera of the sensors 111 that captures the patient's gait. The therapist-specified actuation strategy may be communicated from the remote therapy system 140 to the wearable stimulation arrays 110a-c over the network 160.

The user device 150 may be a personal computer (PC), a tablet PC, a smartphone, or any suitable device capable of executing instructions that specify actions to be taken by that device. The user device 150 may include a processor (e.g., a central processing unit (CPU), a graphics processing unit (GPU), a digital signal processor (DSP), one or more application specific integrated circuits (ASICs), one or more radio-frequency integrated circuits (RFICs), or any combination of these), a memory, a user interface to receive user inputs or provide outputs to the user (e.g., a visual display interface including a touch enabled screen, a keyboard, microphone, speakers, etc.). The visual interface may include a software driver that enables displaying user interfaces on a screen (or display).

The network 160 may serve to communicatively couple the wearable stimulation arrays 110a-c, the sensor 111, the remote mobility augmentation system 120, the database 130, the remote therapy system 140, and the user device 150. For example, the wearable stimulation array 110a and the remote therapy system 140 are configured to communicate via the network 160. In some embodiments, the network 160 includes any combination of local area and/or wide area networks, using wired and/or wireless communication systems. The network 160 may use standard communications technologies and/or protocols. For example, the network 160 includes communication links using technologies such as Ethernet, 802.11, worldwide interoperability for microwave access (WiMAX), 3G, 4G, 5G, code division multiple access (CDMA), digital subscriber line (DSL), etc. Examples of networking protocols used for communicating via the network 160 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network may be represented using any suitable format, such as hypertext markup language (HTML) or extensible markup language (XML). In some embodiments, all or some of the communication links of the network 160 may be encrypted using any suitable technique or techniques.

Although the components of the system environment 100 are shown as connected over the network 160, one or more components may function without being connected to the network 160. For example, the wearable stimulation arrays 110a-c may function offline when the arrays 110a-c are not able to connect to the network 160. When the arrays 110a-c are able to reconnect to the network 160, they may upload measured movement data and corresponding actuation instructions performed to the remote mobility augmentation system 120 or the remote therapy system 140 via the network 160.

### Wearable Stimulation Array

FIG. 2 is a block diagram of a wearable stimulation array 200, in accordance with at least one embodiment. The wearable stimulation array 200 includes electrodes 201, sensors 202, an electrode multiplexer (MUX) 203, a microcontroller (MCU) 204, a power source 205, communications circuitry 206, and a stimulator 207.

The electrodes 201 deliver electrical stimulation to the user of the wearable stimulation array 200. The electrodes 201 may be transcutaneous electrodes for electrical nerve stimulation. Each electrode may be coupled to one or more pads that contact the user's skin to deliver an electrical signal. The placement of the pads of respective electrodes may be spaced apart such that an actuation applied within a proximity (e.g., at the same muscle) of another actuation does not interfere with the other actuation. Alternatively, the MCU 204 may determine which electrodes to apply the electrical stimulation through based on the locations of the electrodes. For example, if electrode ID nos. 1 and 2 are within an inch of one another and electrode ID no. 3 is four inches from both electrodes 1 and 2, the MCU 204 may determine to choose electrode 3 and one of electrodes 1 or 2 as the cathode electrodes such that the two electrical signals do not interfere with one another. The size of the electrode pad may be sized to decrease the likelihood of causing the user pain during stimulation. For example, an electrode pad contacting the skin may have a 24 millimeter (mm) diameter. In some embodiments, the electrodes 201 includes electrodes for sensing EMG data of the user. The electrodes may be surface or skin electrodes, which are non-invasive and structured to adhere to the surface of the skin without penetrating the skin to determine electrical activity when the user's muscles are stimulated.

The electrodes 201 may include two or more electrodes. In one example, the wearable stimulation array 200 may be a 24-electrode array, where the electrodes 201 consist of 24 electrodes. An electrode may serve as a cathode or an anode. For example, the electrodes 201 may form an 8-electrode array that includes two cathodes and six anodes. Additionally, the electrode may be disconnected or off, where it is unselected to be either an anode or cathode. For example, in an 8-electrode array, six electrodes may function as cathodes or anodes while two electrodes are disconnected and do not serve as either a cathode or anode. In some embodiments, an electrode of the electrodes 201 may be used to sense EMG signals. Each electrode of the electrodes 201 may be associated with an identifier (e.g., an identification number). The identifiers may be used in an actuation instruction to identify which electrodes are used during a given actuation instruction. For example, to stimulate a knee extension, electrode ID no. 1 may be used as an cathode and electrode ID nos. 4 and 5 may be used as an anode. The identifiers may also be used to identify which electrodes are used to perform EMG sensing. For example, electrode ID nos. 2 and 3 may be used to perform EMG sensing while the electrodes in the previous example stimulate a knee extension. The wearable stimulation array 200 may be initialized with a default electrode combinations used for actuation or EMG sensing or may be initialized with combinations modified by the user. For example, after a calibration, the user changes the default combinations of electrodes and the new combination is saved in the memory (e.g., the user profile database 222) of the MCU 204 for subsequent initializations of the wearable stimulation array 200.

The mobility augmentation system 220 (e.g., the actuation coordination module 224) may reconfigure the roles of the electrodes 201. The roles of the electrodes may be reconfigured depending on the movement intended to be stimulated or the feedback provided by the user. For example, to stimulate a knee extension, a first set of electrodes may be configured as anodes and a second set of electrodes may be configured as cathodes. To enable an electrode to alternate between an anode or a cathode, the mobility augmentation system 220 may alternate the direction of the current drawn from the power source 205. In the previous example, the first set of electrodes may be configured as cathodes and the second set of electrodes may be configured as anodes to stimulate a different movement (e.g., a knee flexion). The mobility augmentation system 220 may also adjust the role of an electrode to serve as an EMG sensing electrode. For example, the mobility augmentation system 220 may enable a connection between an electrode and an EMG sensor. In addition to reconfiguring the roles of the electrodes 201, the mobility augmentation system 220 may select which electrodes are activated for each actuation instruction or EMG sensing operation. This selection is further described in the description of the MUX 203.

The electrodes 201 may be configured to operate in a measurement mode and an intervention mode. In the measurement mode, the electrodes 201 measure EMG data associated with one or more muscles and report the measured data to the actuation coordination module 224 of the MCU 204, as discussed below. The actuation coordination module 224 detects, based on the measured EMG data, the occurrence of a hyperexcitability event (e.g., a spastic muscle contraction) and reconfigures the electrodes 201 of the array 200 to operate in an intervention mode and apply a selected intervention signal to reduce the hyperexcitability of the one or more muscles. After the intervention signal is applied, the actuation coordination module 224 reverts the array 200 to a measurement mode in which the electrodes 201 continue to measure EMG signal data, which is analyzed to evaluate the efficacy of the applied intervention signal. The electrodes 201 are therefore reconfigurable and can operate in a measurement mode or an intervention mode based on instructions from the actuation coordination module 224.

The electrodes 201 are also configured to deliver both afferent and efferent signals to the user of the wearable stimulation array 200 where the afferent signals are transmitted from sensory receptors or organs toward the central nervous system (e.g., to the brain or spinal cord) and provide information about the state of the body and its external environment (e.g., indicating muscle tension, pressure, temperature, etc.), and the efferent signals (characterized as functional motor activation pulses) are transmitted from the central nervous system towards the body (e.g., to the muscles or nerves) and trigger a response or action based on the information received via afferent signals.

In one embodiment, afferent signals are applied upon initialization of the array 200 to establish a baseline stimulation and maintain a constant interaction with the user's neuromuscular system. If muscle spasticity is detected, the actuation coordination module 224 modifies the set of afferent signals to suppress the detected spasticity, e.g., by increasing the amplitude or frequency of the afferent signals or altering the signal pattern.

A set of efferent signals are selected and delivered based on an identified intended movement of the user. As discussed above, the wearable stimulation array 200 monitors a user's movement to determine intended movement, e.g., using EMG sensors capable of detecting specific muscular or neural patterns that correlate with an intention to move. For example, a set of sensing electrodes may be placed at the shank of the user's right leg to measure the intended movement before and during a gait. Responsive to detecting signals indicative of intended movement, the calibration module 225 selects a set of efferent signals to facilitate the intended movement. In various embodiments, as discussed below, the efferent signal selection is based on one or more of predetermined signal-movement pairings, learned pairings through machine learning algorithms, and user feedback. The selected efferent signals are applied in an interleaved fashion with the afferent signals, thereby facilitating the intended movement (using the efferent signals) and maintaining the baseline neuromuscular interaction or suppressing detected spasticity (using the afferent signals).

The wearable stimulation array 200 may be coupled to an article of clothing for routine use. For example, the wearable stimulation array 200 may be incorporated into a legging such that a set of configurable electrodes (i.e., the electrodes 201) contacts a leg of the user. In another example, the array 200 is coupled to a sock or a shoe insole such that the set of configurable electrodes contacts a foot of the user. The wearable stimulation array 200 may have various wearable form factors such as exoskeletons, modular electrode straps, leggings, foot pressure beds, any wearable form factor suitable for targeting a particular muscle group on a user's body, or a combination thereof.

The sensors 202 measure the user's movement or body measurements related to movement (e.g., heart rate or respiration rate affected by movement). The movement can be measured before, during, or after application of an actuation (e.g., electrical stimulation). Movement measured before actuation may be used to determine which actuation instruction to enable. Movement measured during or after the application of the actuation may be used to score the applied actuation. The sensors 202 may be one or more of a microelectromechanical systems (MEMS) device, IMU, pressure sensor bed, EMG sensor, heart rate sensor, force sensor, or any suitable device for measuring kinetic or kinematic signals produced by a muscle. The sensors 202 may include an EMG sensor, which may include dedicated electrodes (i.e., separate from the electrodes 201) for collecting EMG data, or the wearable stimulation array 200 may obtain EMG data from the electrodes 201. The sensors 202 may include a galvanic skin sensor, which may include dedicated electrodes for measuring changes in sweat gland activity on the skin or may use the electrodes 201 to collect the galvanic skin response data.

The sensors 202 may be located at various locations on the user's body. For example, a pressure sensor bed may be placed in the user's right shoe to measure the user's right foot pressure as the user completes a gait. And, as discussed above, sensing electrodes placed at the shank of the user's right leg can measure the intended movement data before and during the gait. The sensors 202 may be communicatively coupled to the MCU 204 to provide the measured data for determining or optimizing actuation instructions applied by the wearable stimulation array 200. In some embodiments, the locations of the sensors 202 include the joints of the body (e.g., ellipsoid joint and saddle joint). For example, the sensors 202 may measure movement at the ellipsoid and saddle joints IMU's to determine the quality of a user's grip (e.g., how far the user is able to close their hand into a fist).

The sensors 202 may include a sensor that is not co-located with the wearable stimulation array 200 (e.g., the sensors 111). For example, the sensors 202 may include a camera directed at the user and configured to capture image data of the user's movements. The camera may be communicatively coupled to the wearable stimulation array 200 to provide the image data to the MCU 204 (e.g., to the mobility augmentation system 220), which determines an actuation signal to help stimulate the movement depicted in the image or expected to follow the movement depicted in the image. In another example, the sensors 202 may include a sensor to measure strength of a user's grip such as a handgrip dynamometer. The dynamometer may be communicatively coupled to a wearable stimulation array that is worn at the user's hand or forearm, and the measurements from the dynamometer and sensors at the array may be used to adjust actuation instructions to assist the user in gripping objects.

The MUX 203 enables the wearable stimulation array 200 to select a particular combination of the electrodes 201. The selection of electrodes may be performed over time, over particular phases of a movement, any suitable division of stages that may require a different combination of electrodes per stage, or a combination thereof. Although a single MUX is shown in FIG. 2 to maintain clarity in the illustration, the wearable stimulation array 200 may include more than one MUX that functions similar to the MUX 203. The MUX 203 may be coupled to the power source 205, a sensor of the sensors 202 (e.g., an EMG sensor), the MCU 204, the electrodes 201, and the stimulator 207 that applies afferent and efferent signals, as discussed below. The MUX 203 may receive selection signals (e.g., s₁, s₂, etc.) generated by the MCU 204. For example, the actuation coordination module 224 may determine an actuation instruction to apply to stimulate a movement, where the actuation instructions specify a combination of electrodes. The MCU 204 may enable the specified combination to be selected by activating a corresponding combination of the selection signals input to the MUX 203. In one embodiment, as discussed below, the MCU 204 drives the MUX 203 to determine the role of the electrodes 201 between sensing and stimulating based on the user-specific movement model 229.

To use the MUX 203 to select a combination of electrodes 201 that varies over time, the MCU 204 may output a corresponding combination of selection signals that changes over time. The selection of various electrodes over time may be applicable to stimulate a sequence of movements known to occur in the sequence (e.g., a gait cycle). For example, a gait cycle's swing phase can begin at a toe-off, proceed to a mid-swing, and end with a terminal swing. The MCU 204 may access default actuation instructions to calibrate the user's swing phase movements, where each default instruction includes one or more corresponding selection signals to activate the electrodes 201. For example, in an 8-electrode array, there may be two MUX's each having two selection signals s₁ and s₂. To stimulate the toe-off, the MCU 204 may provide selection signal values of 0 and 1 for the respective selection signals s₁ and s₂ to a first MUX to select electrode ID no. 2 as the cathode, where the first MUX is coupled to electrodes 1-4. Further, the MCU 204 may provide selection signal values of 0 and 1 for the respective selection signals s₁ and s₂ to a second MUX to select electrode ID no. 6 as the anode, where the second MUX is coupled to electrodes 5-8. Following the toe-off stimulation, the MCU 204 may change the selection signal values provided to the two MUX's to stimulate the mid-swing movement and finally, may further change the selection signal values to stimulate the terminal swing.

The MCU 204 represents one or more processors such as a microprocessor, a central processing unit, or the like. More particularly, the processing device may be complex instruction set computing (CISC) microprocessor, reduced instruction set computing (RISC) microprocessor, very long instruction word (VLIW) microprocessor, or a processor implementing other instruction sets, or processors implementing a combination of instruction sets. The MCU 204 may also be one or more special-purpose processing devices such as an application specific integrated circuit (ASIC), a field programmable gate array (FPGA), a digital signal processor (DSP), network processor, or the like. The MCU 204 may be configured to execute instructions for performing the operations and steps described herein.

The MCU 204 hosts and executes a mobility augmentation system 220, which includes software modules such as an initialization module 223, an actuation coordination module 224, a calibration module 225, a GUI module 226, and a model training engine 227. The mobility augmentation system 220 includes models for determining actuation (e.g., electrical stimulation) instructions such as a general movement model 228 and a user-specific movement model 229. The mobility augmentation system 220 includes databases such as an actuation instruction database 221 and a user profile database 222. The mobility augmentation system 220 may have alternative configurations than shown in FIG. 2, including different, fewer, or additional components. For example, one or more of the databases 221 or 222 may be stored remotely rather than on a memory of the MCU 204 at the wearable stimulation array 200 (e.g., contents stored in the database 130) and may be accessible through the network 150. In another example, an additional report generation module may generate a report of the applied actuation and the monitored movement data associated with the actuation and provide the report to the remote therapy system 140.

The actuation instruction database 221 stores actuation instructions for enabling the wearable stimulation array 200 to stimulate a user's movement. An actuation instruction may specify an actuation type, duration of actuation, location on a wearable stimulation array at which the actuation is to occur, any suitable parameter of the actuation (e.g., amplitude, frequency, etc.), or combination thereof. An actuation types classifies the actuation into a manner of actuation such as electric, mechanic, haptic, audio, visual, pneumatic, hydraulic, or a combination thereof. The duration of actuation may vary from short (e.g., ten milliseconds) to long (e.g., ten seconds) durations. The duration may vary based on the actuation type. For example, electrical stimulation may last shorter than audio actuation. The location of actuation may indicate which hardware components of the wearable stimulation array output or apply the actuation. For example, the location of actuation may be a particular electrode of the electrodes 201.

In a first example, an actuation instruction specifies that an electrical signal is enabled from a first set of electrodes of the electrodes 201 to a second set of electrodes of the electrodes 201. The actuation instruction may further specify that the electrical signal is to have a duration of 0.5 seconds, a rectangular pulse with a frequency of 100 Hz and an amplitude of 20 mA, and a location of actuation includes the first set of electrodes designated as cathodes (e.g., electrode ID no.'s 1 and 8) and the second set of electrodes designated as anodes (e.g., electrode ID no.'s 2-7).

Actuation instructions may include a combination of different types, durations, and locations. In a second example, an actuation instruction specifies that a first electrical signal is enabled from a first set of electrodes (e.g., electrode ID no. 1) to a second set of electrodes (e.g., electrode ID no.'s 2-4). In addition, actuation instructions of the second example also specify that a second electrical signal is enabled from a third set of electrodes (e.g., electrode ID no. 8) to a fourth set of electrodes (e.g., electrode ID no.'s 5-7). Further yet, the actuation instructions of the second example specify that a haptic actuation (e.g., a vibration) occurs after the electrical stimulation finishes.

Actuation instructions may involve multiple wearable stimulation arrays. In a third example, two wearable arrays may be communicatively coupled over a network to receive instructions that enable simultaneous stimulation at the two arrays. The simultaneous stimulation may be identical or different. For example, the two arrays may be located such that they contact both of the user's shanks. To assist the user with a jumping motion, which may require identical stimulation at both shanks, the two arrays may be instructed to perform the same actuation at the same time. To assist the user with a walking motion, which may require stimulation that is similar in various aspects except for timing, a first array at the user's left leg may be instructed to perform an actuation first and the second array at the user's right leg may be instructed to perform the same actuation after sensors at the first array determine that the stimulated movement at the left leg is finished and transmit a notice to the second array. The instructions may be generated by a mobility augmentation system onboard an MCU of one of the stimulation arrays and communicated to the other array's MCU via a network. Alternatively or additionally, the instructions may be generated by a remote mobility augmentation system at a cloud-based server and communicated to both arrays via a network.

The user profile database 222 stores information regarding one or more users. The users may be users of the wearable stimulation array having memory to store the user profile database 222. In some embodiments, information of users of the wearable stimulation arrays is transmitted to the user profile database 222, which is located at a remote server such as the remote mobility augmentation system 120. The user profile database 222 may include user information such as body measurements (e.g., height, weight, body mass index, body temperature, heart rate, galvanic skin response, etc.), movement measurements (e.g., walking pace, steps taken, elevation gain, exercises performed), and measurements of muscle hyperexcitability and corresponding applied intervention signals. Such user information may be provided by the user manually (e.g., the user entering their height) or tracked by a wearable device such as the stimulation array described herein or a wearable fitness tracker (e.g., a smartwatch).

The user information stored in the user profile database 222 may also be tracked by sensors (e.g., the sensors 202 of the wearable stimulation array 200). For example, the user profile database 222 may store a record of the movement data representing stimulated movement of the user by the wearable stimulation device 200. In some embodiments, the user profile database 222 stores feedback from the user indicating a measure of approval of the stimulated movement. The feedback may be inferred through measurements taken by the wearable stimulation array 200 or manually provided by the user. For example, the wearable stimulation array 200 measures and compares stimulated movement from two different actuation instructions intended to stimulate the same movement (e.g., a dorsiflexion). The wearable stimulation array 200, which may be located at the user's shoe, measures kinetic movement data representing the dorsiflexions of differing qualities stimulated by the two actuation instructions. The wearable stimulation array 200 compares the kinetic movement data of the stimulated movements to kinetic movement data of a neurotypical dorsiflexion and determines that the first actuation instruction stimulated movement that is more similar to neurotypical movement than the second actuation instruction stimulated.

In another example, a user manually provides feedback that is stored in the user profile database 222. The wearable stimulation device 200 may be communicatively coupled to a user device (e.g., the user device 150) through which the user can provide feedback indicating a measure of comfort or effectiveness of the actuation by the wearable stimulation device 200. In some embodiments, user-provided feedback stored in the user profile database 222 includes feedback indirectly provided by the user through a user interface. For example, the user adjusts the actuation instruction via a GUI on a user device communicatively coupled to the wearable stimulation array 200. The user profile database 222 may store the user's chosen actuation modification and link the modification to the stimulated movement such that subsequent array-actuated stimulations of the movement can reference the user's modification stored in the database 222.

The initialization module 223 initializes the wearable stimulation array 200. The initialization module 223 may help conserve power of the wearable stimulation array 200 that is not necessarily attached to a steady power source (i.e., the array 200 may be a wireless device). For example, the initialization module 223 may pause functions such as sensing the user's muscle firings (e.g., by determining EMG signals via the electrodes 201 or sensing movement data via the sensors 202) after the module 223 determines that a user is not wearing the wearable stimulation array 200 and resume the functions after the module 223 determines that the user has resumed wearing the array 200. The initialization module 223 may determine whether the user is wearing the array 200 using sensors (e.g., the sensors 202 or a remote sensor such as a camera communicatively coupled to the MCU 204). For example, the initialization module 223 uses data from a heart rate sensor of the sensors 202 to determine that no heart rate has been sensed for more than a predetermined time threshold (e.g., 10 seconds) and thus, the user has removed the wearable stimulation array 200. Similarly, the module 223 may determine that a heart rate has been detected a minimum number of times (e.g., 5 times) within a threshold period of time (e.g., 8 seconds) and thus, the user has worn the wearable stimulation array 200.

The initialization module 223 additionally configures the mode of operation of the electrodes 201 of the wearable stimulation array 200. In one embodiment, the initialization module 223 configures the array 200 to operate initially in a measurement mode in which the electrodes 201 measure EMG data (e.g., an EMG signal) from one or more muscles of the user. As discussed in more detail below, responsive to the actuation coordination module 224 detecting the occurrence of a hyperexcitability event (e.g., a spastic muscle contraction) based on the measured EMG data, the array 200 electrodes 201 of the array 200 are reconfigured to operate an intervention mode in which the electrodes 201 apply an intervention signal to the one or more muscles to reduce or eliminate the identified muscle hyperexcitability. Default configuration of the array 200 in the measurement mode ensures that EMG measurements are captured and analyzed upon initialization of the array 200 to enable detection of a hyperexcitability event.

The initialization module 223 is further configured to select baseline afferent signals for application by the electrodes 201 upon initialization of the wearable stimulation array 200. In one embodiment, the selected baseline afferent signals are customized based on user-specific information (e.g., age, weight, health condition, typical activities performed by the user, user feedback, etc.) while in other embodiments, the electrodes 201 apply a standard configuration of initial afferent signals. As discussed in more detail below, the afferent signals are dynamically modified in response to detection of muscle spasticity or rigidity and are interleaved with efferent signals upon detection of an intended movement.

The actuation coordination module 224 enables or disables actuation applied through a wearable stimulation array. The actuation coordination module 224 can determine likely movements (e.g., ankle dorsiflexion) to determine a corresponding actuation to apply. The actuation coordination module 224 may determine a movement based on EMG data, IMU data, foot plantar pressure signals, a context in which the movement occurs, any suitable data through which movement can be inferred, or a combination thereof. The module 224 may use one or more of the aforementioned data types to determine a movement of a set of movements with which a wearable stimulation array is configured to assist the user. Example movements may include those within a gait cycle which as a heel strike, loading response, mid-stance, terminal stance, pre-swing, toe-off, mid-swing, and terminal swing. The term “movement” used herein may refer broadly to an activation of a particular muscle group to move or maintain a position. For example, standing may be a type of movement that the wearable stimulation array described herein may assist in despite minimal to no movement relative to other movements such as walking or running.

The actuation coordination module 224 can determine a movement using EMG data to enable a corresponding actuation instruction. For example, upon identifying, based on measured EMG signals, a muscle firing event corresponding to a kinematic signal associated with a toe lift of the user's contralateral foot, the module 224 may apply an actuation instruction associated with a toe lift from a contralateral foot. The actuation coordination module 224 may receive EMG data from electrodes of the wearable stimulation array that are configured to sense EMG signals of muscle firing events. Alternatively or additionally, the user may be wearing electrodes (e.g., multiple wearable stimulation arrays) at various locations on the user's body and the wearable stimulation array 200 may receive EMG signals sensed from electrodes of a different device or wearable stimulation array. For example, a user may be experiencing a limp in their right leg, wears wearable stimulation arrays on both their left and right legs, uses a first array on their left leg to measure EMG signals corresponding to a gait cycle in the left leg, and uses a second array on the right leg, which receives the measured EMG signals from the first array to stimulate electrical signals to assist in the gait cycle in the right leg.

The actuation coordination module 224 can determine a movement using IMU data to enable a corresponding actuation instruction. For example, an IMU sensor of the sensors 202 may provide kinematic signals associated with various stages of a gait cycle to the actuation coordination module 224. The module 224 may track the various stages against the predefined order of stages in a gait cycle (e.g., toe-off precedes mid-swing) to determine that a movement is likely to occur based on currently measured kinematic signals. For example, the module 224 determines that a toe-off movement is occurring using data from the IMU sensor and determines that the user is likely to perform a mid-swing movement. Based on this determination, the module 224 may apply an actuation instruction to enable the mid-swing movement. The actuation coordination module 224 may receive IMU data from the sensors 202 or a user device co-located with the user (e.g., a smartphone or smartwatch).

The actuation coordination module 224 can determine a movement using foot plantar pressure signals to enable a corresponding actuation instruction. For example, the actuation coordination module 224 may access foot plantar pressure signals from pressure sensors of the sensors 202 contacting the sole of a user's foot. The actuation coordination module 224 may use the accessed pressure signals to determine that the user is likely attempting to perform ankle plantarflexion (e.g., when jumping or standing on the tips of the toes to reach an object). In response, the module 224 may enable an actuation instruction to assist in the ankle plantarflexion. For example, the module 224 may enable actuation instructions that apply electrical stimulation via electrodes at a wearable stimulation array contacting the calve muscles to assist in the ankle plantarflexion. This wearable stimulation array contacting the calve muscles may be the same array sensing the foot plantar pressure signals or a different array that is communicatively coupled. The sensors 202 may be part of a foot pressure bed, sock, legging, or any suitable form factor for contact with the sole of the user's foot.

The actuation coordination module 224 can determine a context (e.g., when and where) to enable a corresponding actuation instruction. For example, the actuation coordination module 224 can determine contexts in which the user is likely intending to perform an ankle dorsiflexion, which may be referred to herein by “dorsiflexion” unless specified otherwise by context, such as while the user is walking, dancing, or preparing to stand from a seated position. The actuation coordination module 224 can determine a context in which the stimulated movement is to occur based on the user (e.g., user information such as body measurements, activity measurements, or the user's past or upcoming schedule), a location of the wearable stimulation array on the user's body, a time of day, or a location of the user. The actuation coordination module 224 may determine context using information stored in the user profile database 222. For example, the actuation coordination module 224 may access the user's schedule stored in the database 222 indicating that the user is scheduled to go to a dance class at 5:30 PM, determine that the time of day is 5:35 PM, and determine the context that the user is likely at a dance class and may be more likely to perform movements (e.g., knee and ankle flexions and extensions) than a context such as scheduled to be at a job at a desk where knee and ankle movement is minimal.

The actuation coordination module 224 may access a movement model (e.g., the general movement model 228 or the user-specific movement model 229) to enable actuation at the wearable stimulation array 200 (e.g., an electrical signal from one electrode to another) and stimulate movement by the user. For example, the module 224 accesses the general movement model 228 to enable actuation when a user whose movement data and actuation preferences (e.g., feedback scores for applied actuation) have not yet been recorded to provide more customized actuation. In another example, the module 224 accesses the user-specific movement model 229 to enable actuation that is more tailored to the user's body than the general movement model 228 through retraining of the model 229 using user feedback and measured stimulated movement to gauge the level of comfort or efficacy of the applied actuation instructions.

In some embodiments, the actuation coordination module 224 may stimulate the movement by enabling a first electrical signal from a first electrode to a second electrode and a second electrical signal from a third electrode to a fourth electrode. In some embodiments, a ratio of a pulse width of the first electrical signal to a pulse width of the second electrical signal is predetermined. This predetermined ratio may be referred to as a “proportional steering ratio.” In one example, the actuation coordination module 224 determines a first electrode configuration where a first electrical signal of a particular frequency, pulse width, and amplitude is delivered using the electrodes in the first configuration. In this example, the actuation coordination module 224 also determines a second electrode configuration where a second electrical signal is delivered using the electrodes in the second configuration. The actuation coordination module 224 may determine pulse width of the second electrical signal using a predetermined proportional steering ratio. The ratio may be 1:4, where the pulse width of the first electrical signal is 20% and the pulse width of the second electrical signal is 80%. In this way, the actuation coordination module 224 can split the pulse width between two electrode configurations according to the proportional steering ratio. This may help increase the density of the wearable stimulation array without having to add additional physical electrodes.

The actuation coordination module 224 may determine a level of fatigue experienced by the user during movement using the measured user activity data. For example, the sensors 202 measure movement data (e.g., kinetic signals, kinematic signals, pressure signals) of the user's movement for a particular movement. The module 224 may track the amplitude of the signals of the movement data compared to a baseline signal profile of the movement. For example, during calibration, the calibration module 225 may measure and record a baseline signal profile for an ankle dorsiflexion using the IMU sensor of the sensors 202 that measures amplitude over time of the kinematic signal associated with the dorsiflexion. The module 224 may determine that the resulting dorsiflexion assisted by a particular actuation instruction is decreasing in amplitude or the duration of the movement is increasing as the user performs their daily routine due to the user's fatigue. The module 224 may determine that the amplitude has decreased by 0.5% between an earlier and later dorsiflexions or the duration has increased by 1% between the earlier and later dorsiflexions. This percentage may be proportional to a level of fatigue experienced by the user and used to adjust the actuation instruction. For example, the module 224 can access predefined levels of fatigue (e.g., a scale from 1 to 5) where a range of within ±5% variation from the baseline signal profile may correspond to the minimum level of fatigue (e.g., a level of 1), within ±25% variation from the baseline signal profile may correspond to a next higher fatigue level (e.g., level 2), within ±50%, within ±75%, and greater than ±100% variation for respective, subsequent levels.

In some embodiments, the actuation coordination module 224 may determine a level of fatigue using EMG signals measured by the sensors 202. The actuation coordination module 224 may determine a frequency response of the electroactivity in the EMG signals and determine that the frequency of electroactivity is lower (e.g., on average) than the frequency response of a baseline frequency response determined using EMG signals measured when the user was rested.

The actuation coordination module 224 may also adjust the actuation instruction (e.g., a configured power of electrical stimulation applied) based on the determined level of fatigue. Continuing the previous example, the module 224 may adjust the actuation instruction according to predefined conditions corresponding to the respective variation percentages from baseline. For example, if the kinematic signal amplitudes of a user's dorsiflexions as measured by an IMU sensor have decreased by 60% from the baseline profile, the module 224 may adjust the corresponding actuation instruction for dorsiflexion by applying a gain factor of 1.6 to the electrical stimulation applied to the user's shank.

The actuation coordination module 224 may process image or video data captured by a remote sensor (e.g., a camera communicatively coupled to the wearable stimulation array 200). For example, a user installs cameras throughout their home (e.g., as part of an assisted living or a remote care environment), where the cameras capture the user walking and transmit the captured images to the wearable stimulation array 200. The module 224 may perform image processing or apply machine learning on the captured data to recognize the position of the user's legs over time and determine a likely, upcoming movement in the user's gait cycle. The module 224 may identify each movement in a gait cycle. For example, the module 224 can determine that a terminal stance is likely to be the upcoming movement following a mid-stance identified in the latest image data received from the camera. In this way, the module 224 determines that the mid-stance depicted in the image data is a movement within the set of movements of a gait cycle.

In some embodiments, the actuation coordination module 224 determines that the user is performing a movement based on the captured image data, movement data captured from an IMU sensor or a foot pressure sensor of the sensors 202, or EMG data captured from electrodes of the electrodes 201. For example, the module 224 may weigh the output of the image processing with the output of a movement model trained to determine an actuation instruction based on movement data measured by the IMU sensor, where the model output includes an intermediate determination of the movement likely reflected in the movement data. The module 224 may weigh the output of the image processing lower than the IMU sensor, and may adjust this weight based on the user feedback. For example, if the user's feedback indicates that actuation instructions determined using IMU sensor data is unsatisfactory (e.g., causing them discomfort or is not effective in assisting with movement), the module 224 may decrease the weight of the output of the movement model or increase the weight of the output of the image processing. Alternatively or additionally, the model training engine 227 may also use this user feedback to retrain the movement model trained on IMU sensor data.

In some embodiments, the actuation coordination module 224 disables actuation. For example, the actuation coordination module 224 may receive user feedback (e.g., via a GUI at a user device communicatively coupled to the wearable stimulation array 200) to stop the actuation and the module 224 will pause or end the actuation being applied.

The actuation coordination module 224 also coordinates the reconfiguration of the array 200 between the initial measurement mode (also referred to as the “first measurement mode”) set by the initialization module 223 and an intervention mode in which an intervention signal is applied responsive to detecting the occurrence of a hyperexcitability event. To do so, the actuation coordination module 224 monitors measured EMG signal data reported by the electrodes 201 to identify anomalous activity data representative of muscle hyperexcitability. For example, a spike in EMG signal amplitude due to increased muscle activity and the temporal extent of such spikes relative to normal muscle activity may indicate spastic contractions or hyperexcitable events. Similarly, hyperexcitability may manifest as frequent, irregular muscle spasms reflected as erratic, repeating patterns in the EMG signal data.

The actuation coordination module 224 compares the EMG signal data collected during the first measurement mode (e.g., as determined by the amplitude, frequencies, and/or durations of the signals) to at least one hyperexcitability threshold. Because signal characteristics may vary based on muscle type or location and the user's personal physiology, in various embodiments, different threshold values may be used based on a type of muscle from which the EMG data is measured or a type of hyperexcitability event. For example, a higher threshold value may be applied for a first type of muscle showing stronger contractions (and hence, higher EMG signals) than to a second type of muscle showing lower contractions (and hence, lower EMG signals). Moreover, in some embodiments, multiple thresholds may be applied to determine the scope or severity of a hyperexcitability event. For example, EMG signal data exceeding a first, lower threshold might indicate a mild hyperexcitability event while signal data exceeding a second, higher threshold might indicate a severe hyperexcitability event.

Responsive to the EMG signal data exceeding at least one hyperexcitability threshold, the actuation coordination module 224 reconfigures the electrodes 201 of the array 200 to operate in an intervention mode in which the electrodes 201 apply an intervention signal to the one or more muscles for which a hyperexcitability is detected. The actuation coordination module 224 determines an intervention signal for application by the set of electrodes 201 to the one or more muscles based on factors including the type of hyperexcitability event detected, the severity of the event, and the affected muscle. For example, the actuation coordination module 224 might instruct the electrodes 201 to apply an intervention signal with high amplitude or different frequency patterns to a more severe or prolonged hyperactive muscle event than to a milder event.

As discussed above, a user may provide feedback indicating a measure of comfort or effectiveness of an intervention signal applied by the wearable stimulation device 200. The feedback data is stored in a user profile in the user profile database 222. In some embodiments, the actuation coordination module 224 considers stored user feedback in determining an appropriate intervention signal. Moreover, as discussed below, the model training engine 227 re-trains a general movement model 228 to customize the model to the user's motions, and the re-trained model is the user-specific movement model 229. The user-specific data training set may include data indicating relationships between EMG signal patterns and successful interventions such that output from the user-specific movement model 229 may be used as input to the actuation coordination module 224 to identify an appropriate intervention signal for a hyperexcitability event. As a result, the selection and calibration of intervention signals may be improved over time, becoming more tailored and responsive to the user's needs. Moreover, in instances where a previous intervention did not successfully reduce or eliminate the identified muscle hyperexcitability, the actuation coordination module 224 may identify a different type of intervention signal or adjust the existing signal parameters (e.g., the frequency or the amplitude of the intervention signal).

Upon identifying an intervention signal to apply to the one or more muscles, the actuation coordination module 224 instructs the electrodes 201 to transmit the intervention signal. After the intervention signal is applied, the actuation coordination module 224 reconfigures the electrodes 201 to return the array 200 to the measurement mode (e.g., a “second measurement mode”) in which EMG signal data is measured and evaluated again. The actuation coordination module 224, in conjunction with the calibration module 225, also adjusts a subsequent intervention signal based on measurements captured by the electrodes 201 during the second measurement mode, as discussed in more detail below. In this way, the actuation coordination module 224 and calibration module 225 operate a feedback loop in which the efficacy of a first intervention signal is evaluated using EMG measurements captured after the first intervention signal is applied (e.g., during the second measurement mode) and a subsequent intervention signal is determined and applied based on the evaluated efficacy.

The calibration module 225 also calibrates applied intervention signals based on detected muscle hyperexcitability. As discussed above, after a first intervention signal is applied, the actuation coordination module 224 reconfigures the electrodes 201 to place the array 200 in the second measurement mode. The calibration module 225 evaluates second measured EMG data gathered by the electrodes 201 during the second measurement mode (i.e., after the first intervention signal is applied) to evaluate the efficacy of the intervention signal by determining whether the intervention signal achieved a desired reduction of muscle hyperexcitability. In one embodiment, the calibration module 225 evaluates the applied intervention signal by comparing the original EMG data gathered during the first measurement mode (e.g., as determined by the amplitude, frequencies, and/or durations of the signals) with the second, post-intervention EMG signal data gathered during the second measurement mode. If the calibration module 225 determines, based on the comparison, that the amplitude, frequency, duration, or other relevant EMG parameters have decreased by more than a threshold amount, the calibration module 225 determines that the applied intervention signal was successful in reducing the muscle hyperexcitability. Conversely, if the comparison indicates that the intervention signal did not reduce the muscle hyperexcitability by at least the threshold amount, the calibration module 225 determines that the intervention signal was unsuccessful and that a second intervention signal should be applied. Moreover, as discussed above, in some embodiments, user feedback may be used to evaluate whether an applied intervention signal was successful. For example, a user may provide feedback about perceived improvements in muscle hyperexcitability or associated issues (e.g., pain reduction, improved motion, etc.).

If the calibration module 225 determines that the applied intervention signal was successful, the actuation coordination module 224 continues to operate the array 200 in the measurement mode until a subsequent hyperexcitability event of the same or a different type is detected. That is, the actuation coordination module 224 instructs the electrodes 201 to continue measuring and reporting EMG data, and the actuation coordination module 224 continues to compare the received data to the at least one hyperexcitability thresholds to detect the occurrence of a hyperexcitability event. During this measurement mode, an intervention signal is not applied.

In some embodiments, if a subsequent hyperexcitability event is detected, the calibration module 225 determines a different intervention signal to apply even if the first intervention signal is classified as successful. For example, if, during a subsequent monitoring mode, the actuation coordination module 224 determines that a second hyperexcitability event has occurred, rather than reapplying the same intervention signal successfully applied in response to the first hyperexcitability event, the calibration module 225 may identify a second intervention signal of shorter duration or reduced amplitude to attempt to successfully reduce the muscle hyperexcitability with less intervention than was applied in response to the first hyperexcitability event. In this context, if the second intervention signal is determined to be unsuccessful or less successful than the first intervention signal (e.g., the second intervention signal does not reduce muscle hyperexcitability by at least a threshold amount or reduces muscle hyperexcitability by less than the first intervention signal), the calibration module 225 may determine to reapply the first intervention signal or further adjust the first intervention signal to an amplitude or frequency less than the first (successful) intervention signal but greater than the second (unsuccessful) intervention signal.

If the calibration module 225 determines that a first applied intervention signal was not successful (i.e., the first intervention signal did not reduce the muscle hyperexcitability by at least a threshold amount), the calibration module 225 selects a second intervention signal to apply. The calibration module 225 selects the second intervention signal based at least in part on the second EMG data gathered during the second measurement mode. For example, the calibration module 225 may adjust (e.g., increase) the amplitude of the intervention signal, alter (e.g., increase or decrease) the pulse frequency, adjust the pulse width and overall duration of the intervention signal, reconfigure the electrodes 201 acting as anodes or cathodes, manipulate the overall electrode placement, or otherwise adjust the waveform shape or pattern of the intervention signal to improve the efficacy of the intervention.

After the calibration module 225 selects a second intervention signal, the actuation coordination module 224 reconfigures the array 200 to operate in a second intervention mode such that the electrodes 201 apply the second intervention signal to the one or more muscles experiencing the hyperexcitability event. After the second intervention signal is applied, the actuation coordination module 224 may revert the electrodes 201 of the array 200 to the measurement mode (i.e., a third measurement mode) to evaluate the efficacy of the second intervention signal using the method described above. The actuation coordination module 224 and calibration module 225 may continue to evaluate EMG data during measurement modes and adjust and apply intervention signals during intervention modes until the muscle hyperexcitability is reduced by at least a threshold amount. In this way, the actuation coordination module 225 and calibration module 225 operate a feedback loop to adjust applied interventions based on EMG signal data measured by the electrodes 201.

The actuation coordination module 224 also detects a measure of spasticity of one or more muscles of the user by monitoring EMG signals produced by the muscles in real time. In one embodiment, the actuation coordination module 224 analyzes incoming EMG signals from the electrodes 201 and identifies patterns in the signals to detect spastic muscle behavior. For example, spasticity may be indicated by one or more of a persistent level of electrical activity higher than a baseline, irregular and high-frequency bursts of electrical activity signaling involuntary muscle contractions, or synchronized firing of motor neurons and muscle fibers. The measure of spasticity of the one or more muscles may be represented as EMG signal variance (where increased variance indicates higher levels of spasticity), frequency of abnormal, high-frequency signal peaks within a specified time period (where a higher frequency represents increased muscle spasticity), or a degree of EMG signal synchronization (where higher levels of synchronization indicate spasticity). The actuation coordination module 224 calculates a measure of spasticity using one or more of these metrics.

The calibration module 225 selects a set of afferent signals to apply based on the measure of spasticity calculated by the actuation coordination module 224. In one embodiment, the calibration module 225 compares the measure of spasticity to at least one spasticity threshold to determine a level of severity of the spasticity. For example, a measure of spasticity above a first threshold might indicate minor spasticity, requiring lower amplitude or frequency of afferent signals, while a measure of spasticity above a second threshold might indicate severe spasticity, such that the calibration module 225 selects a set of afferent signals at a higher amplitude or frequency or a different pattern of afferent signals.

In another embodiment, the calibration module 225 uses a machine-learning model, such as the general movement model 228 or the user-specific movement model 229 to select the set of afferent signals. For example, the detected measure of spasticity may be provided as input, and the model may compute parameters (e.g., strength, frequency, duration, etc.) of an optimal set of afferent signals to reduce or eliminate the spasticity. Where the calibration module 225 uses machine learning techniques to select the set of afferent signals, the model may be retrained based on updated training data including measures of success of applied afferent signals, user feedback, and the like.

The actuation coordination module 224 and calibration module 225 operate, in some embodiments, a feedback loop to dynamically adjust applied afferent signals based on real-time spasticity detection. For example, the actuation coordination module 224 uses the electrodes 201 to monitor the EMG signals from the one or more muscles and can calculate updated spasticity measures based on the magnitude of detected changes. Updated spasticity measures may be sent to the calibration module 225, which selects an updated set of afferent signals, e.g., by adjusting the amplitude, frequency, or temporal pattern of the signals based on the updated spasticity measures. As the electrodes 201 of the array 200 apply the adjusted afferent signals, the actuation coordination module 224 continues monitoring EMG signals to evaluate the efficacy of these adjustments. If the spasticity continues or the severity of the spasticity changes, further adjustments can be made to the afferent signals to reduce or eliminate the spasticity. For example, where higher levels of muscle spasticity are detected, the calibration module 225 might increase the amplitude or frequency of the afferent signals, providing the sensory system with more input to counter muscle oversensitivity. Conversely, if the muscle spasticity decreases, the calibration module 225 may reduce the amplitude or frequency of the afferent signals to avoid overstimulation.

The actuation coordination module 224 also selects a set of efferent signals based on an intended movement of the user. As described above, the actuation coordination module 224 can determine intended movements of a user based on EMG data, IMU data, foot plantar pressure signals, a context in which the movement occurs, any other suitable data through which movement can be inferred, or a combination thereof. In one embodiment, the electrodes 201 are configured to measure EMG sensor data indicative of intended movement by detecting an electrical change in the tissue of the target muscle (e.g. before and during a gait). The actuation coordination module 224 analyzes the EMG signal data to identify the intended movement and select corresponding efferent signals to facilitate the identified movement.

In various embodiments, efferent signal selection is based on predefined mappings between specific intended movements and corresponding efferent signals or learned pairings through application of machine learning algorithms. Alternatively, the actuation coordination module 224 uses the amplitude and frequency of the measured EMG signals to select the set of efferent signals. User feedback may also be used to select the set of efferent signals, e.g., where a user has indicated a measure of effectiveness of previously applied efferent signals to stimulate the same intended movement. Moreover, in one embodiment, selection of the set of efferent signals also includes identification of a maximum efferent signal amplitude, e.g., as determined by the general movement model 228 or user-specific movement model 229 or based on signal safety limits.

The actuation coordination module 224 also coordinates the interleaving of the sets of afferent and efferent signals over the same time period by instructing the electrodes 201 to sequentially apply the signals in a manner that avoids mutual interference. In one embodiment, the afferent and efferent signals are alternately applied. For example, an afferent signal (a baseline afferent signal or an afferent signal selected based on detected muscle spasticity) could be transmitted to a target muscle first, followed by an efferent signal selected based on a detected intended movement, followed by a subsequent afferent signal, etc. The actuation coordination module 224 monitors the signal frequency of the afferent and efferent signals to prevent signal overlap (which could result in decreased effectiveness of the signals, confused sensory feedback, or discomfort to the user), while allowing tone reduction and functional motor activation pulses to be effectively applied over the same time period.

In one embodiment, the actuation coordination module 224 instructs the electrodes 201 to delay application of a scheduled afferent or efferent signal or to skip a scheduled signal responsive to determining that an upcoming afferent signal and an upcoming efferent signal will overlap or occur within a threshold time period of each other. For example, if an efferent signal selected based on a detected intended movement is determined to coincide too closely with a next scheduled afferent signal, the actuation coordination module 224 instructs the set of electrodes 201 to delay application of the efferent signal by a specified amount of time, ensuring a sufficient gap for both signals to operate effectively. Similarly, if an afferent signal is scheduled to be applied during application of an efferent signal, the actuation coordination module 224 may instruct the electrodes 201 to skip or delay the afferent signal until application of the efferent signal is complete. In this way, the timing and sequencing of the afferent and efferent signals may be dynamically adjusted to ensure effective stimulation and reduction of tone.

The calibration module 225 also calibrates an actuation applied by the wearable stimulation array 200 such that it is customized to the user's body or behavior. The calibration module 225 receives user feedback of movement stimulated by the wearable stimulation array 200 and scores, using the received feedback, the corresponding actuation instruction that contributed to the stimulated movement. The scores determined by the calibration module 225 can be used by the model training engine 227 to retrain a movement model, optimizing subsequent actuation determinations with the user's feedback. Calibration can be performed periodically (e.g., once every week), on-demand, or at the initialization of the wearable stimulation array (e.g., each time the user puts on the wearable stimulation array 200).

The feedback received by the module 225 can include data measured by the sensors 202 and data provided by the user (e.g., using user interfaces through which the user can interact with the wearable stimulation array 200). User feedback through a GUI may be direct feedback (e.g., a score, star rating, thumbs up or down, etc.) or indirect feedback (e.g., adjusting the actuation or stopping the actuation) indicating a level of approval with the actuation provided by the wearable stimulation array 200. User feedback may include the efficacy of the stimulated movement as measured by the sensors 202 and compared with a neurotypical profile of the movement or the user's baseline profile of the movement. For example, the calibration module 225 may determine that the stimulated movement's measured kinematic signals are outside of a ±20% amplitude threshold with the amplitude of a neurotypical profile of the same movement and thus, determine that the stimulated movement is not effective.

In addition to using user-provided feedback to calibrate the applied stimulation, the calibration module 225 may use a target movement to evaluate the stimulated movement and retrain a movement model. The target movement may be neurotypical movement or a baseline movement set by the user. The calibration module 225 may compare the measured stimulated movement to a target movement. For example, when calibrating the wearable stimulation array 200 to help a user achieve a movement that resembles neurotypical movement, the calibration module 225 may compare the stimulated movement to neurotypical movement to determine whether an actuation instruction should be modified.

In some embodiments, the calibration module 225 may receive movement data representing a user's performance of a movement without stimulation. The calibration module 225 may determine a movement progress of the user based on the movements and retrain a movement model using the movement progress. For example, the calibration module 225 receives movement data of a user performing a knee extension once per day without stimulation helping the user. The movement data shows that the user is performing the knee extension closer and closer to the target movement over the weeks. The calibration module 225 may retrain the calibration module 225 to strengthen an association between the applied actuation instruction and the movement data associated with the knee extension.

For each actuation instruction applied during calibration, the calibration module 225 may score the resulting stimulated movement measured by a sensor (e.g., the sensors 202). The module 225 may determine the score based on one or more of received feedback (e.g., feedback directly provided by the user) or the comparison of the measured movement data with neurotypical movement data. For example, the comparison performed by the module 225 indicates that the movement stimulated on a user by the default actuation instruction for a dorsiflexion is weaker than the neurotypical movement. In response, the calibration module 225 scores the default actuation instruction low for that user (e.g., using a number system where a lower number represents poorer performance of the actuation instruction). The module 225 may store the scores in the user profile database 222.

The calibration module 225 may receive feedback from the user indicating a measure of approval of the stimulated movement in response to the use of an accessed model to stimulate a movement of a set of movements by the user using the wearable stimulation array 200. For example, after the actuation coordination module 224 uses the user-specific movement model 229 to stimulate a pre-swing in a gait cycle, the calibration module 225 receives feedback from the user indicating that the stimulation was uncomfortable (i.e., the measure of approval of the stimulated movement is low). The module 225 may calibrate the wearable stimulation array 200 by causing the model training engine 227 to retrain the accessed model to change, for at least the stimulated movement (e.g., the pre-swing) of the set of movements, a component of the actuation instruction such as an electrical signal or the electrodes that operated as anodes or cathodes.

In some embodiments, the model training engine 227 trains or retrains a movement model based on the scoring. For example, the user provides feedback that the actuation instruction caused pain where resulting electrical stimulation was applied, the module 225 scores the actuation instruction low for the corresponding movement intended to be stimulated (e.g., a knee extension), and the model training engine 227 retrains a movement model such that the likelihood of the actuation instruction being selected is decreased for subsequent determinations that the user is likely performing a knee extension.

In some embodiments, the calibration module 225 may begin calibration with a default set of actuation instructions for respective movements. The module 225 may access data representing neurotypical movements, which may be stored in the database 130, 221, or 222. The calibration module 225 compares the measured stimulated movement to a neurotypical signal profile of the corresponding movement. For example, the calibration module 225 compares measured kinetic signals of an ankle dorsiflexion to signals representing a neurotypical ankle dorsiflexion. The module 225 uses the comparison to adjust a default actuation instruction. For example, if the comparison indicates that the stimulated movement is not as strong (e.g., the amplitude of the signals are not as high) as the neurotypical movement, the module 225 may adjust the actuation instructions by changing the electrodes used to apply the electrical stimulation or the amplitude of the electrical signal. During calibration, the wearable stimulation array 200 can measure stimulated movement for various movements and compare the stimulated movement to the corresponding neurotypical data to adjust the actuation for each movement.

The calibration module 225 may adjust a frequency, an amplitude, a pulse width, or any suitable parameter of an electrical signal included within an applied actuation instruction. The calibration module 225 may also adjust the configuration of the electrodes 202, configuring the operation of an electrode to be either a cathode or an anode. These adjustments may occur sequentially such that actuation permutations are iterated through and applied for a user to determine which permutation is preferred. The calibration module 225 may apply each permutation with a pause (e.g., 10 second pause) in between consecutive actuations for a user to provide feedback of the latest actuation applied. For example, the module 225 may, as a first actuation in the iteration, enable a first electrical signal having a frequency of 20 Hz using electrode no. 1 as an anode and electrode no. 2 as a cathode. The module 225 may then pause for ten seconds to allow the user to provide feedback. The module 225 may then apply a second actuation in the iteration where the first electrical signal is applied using electrode no. 2 as an anode and electrode no. 3 as a cathode. The iterative actuations may continue through varying permutations of electrode configurations, with pauses in between each actuation, and then alter the electrical signal by incrementing the frequency by 5 Hz to produce a second electrical signal. The module 225 may then apply this second electrical signal through the same permutations of electrode configurations, pausing between each actuation to receive user feedback of the second electrical signal as applied through a particular electrode configuration. The module 225 can use the received feedback determine a score, if not directly provided by the user, and provide the determined score to the model training engine 227 to retraining the accessed model, calibrating the wearable stimulation array 200.

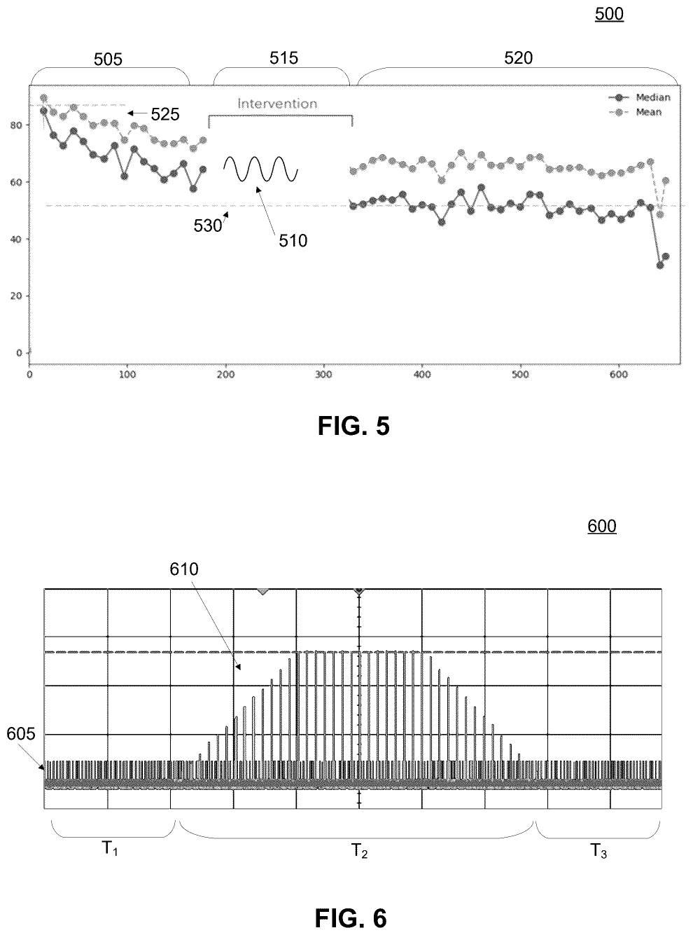

The calibration module 225 may adjust a signal parameter in response to user feedback. For example, the user may use a GUI shown in FIG. 5 to change the frequency or amplitude of electrical stimulation applied. When adjusting a signal parameter, the module 225 may determine indirect feedback provided by the user, as the adjustment may indirectly indicate a low measure of approval with the stimulated movement.

The calibration module 225 may store measured movement data for calibrating the wearable stimulation array 200 or training a movement model. The stored movement data characterizes movement measured by sensors on or coupled to the wearable stimulation array 200. Examples of measured movement data includes kinetic signals from IMU sensors or foot plantar pressure signals from a foot pressure bed. The calibration module 225 may also store measured motor intent data such as EMG signals measured from electrodes of the wearable stimulation array 200. The data may be stored in memory local to the array (e.g., the user profile database 222) or remote to the array (e.g., the database 130). The calibration module 225 may access the stored data to score actuation instructions for corresponding stimulated movements to calibrate the wearable stimulation array. The model training engine 227 may access the stored data to train or retrain a model (e.g., a machine learning model) to determine an actuation instruction. For example, the model training engine 227 may label the measured movement data with a corresponding actuation instruction to generate a training set to train the user-specific movement model 229.

In some embodiments, the data stored by the calibration module 225 may be used to create a movement profile of the user. For example, after the calibration module 225 has finished calibrating the wearable stimulation array 200, the kinetic signals measured when the user performs a corresponding stimulated movement or unstimulated movement may serve as a baseline signal profile of the movement when stimulated or unstimulated, respectively. These baseline profiles can be used to determine, for example, if the user is experiencing fatigue and whether the actuation coordination module 224 should adjust actuation instructions.

The GUI module 226 generates for display a GUI through which the user can provide feedback of the applied actuation instructions or control the wearable stimulation array 200. The GUI may be generated on a user device coupled to the wearable stimulation array 200. The GUI module 226 may display information describing the actuation such as properties of an applied electrical signal and through which electrodes the signal is applied (e.g., the ID numbers of the electrodes serving as the anodes and cathodes). The GUI module 226 may provide an interactive user interface that includes various buttons, toggles, menus, etc. through which a user can adjust the applied actuation. The interactive user interface may also include user inputs for providing feedback.

The model training engine 227 may train a machine learning model in multiple stages. In a first stage, the model training engine 227 may use generalized data representative of measured movement (e.g., kinetic signals, kinematic signals, or EMG signals) collected across one or more users (e.g., a neurotypical population) to train the machine learning model. The model training engine 227 may label the generalized data with an instruction label representative of the actuation instruction that should be applied to assist with the measured movement represented by the generalized data. The model training engine 227 creates a first training set based on the labeled generalized data. The model training engine 227 trains a machine learning model (e.g., the general movement model 228), using the first training set, to determine an actuation instruction to enable using the wearable stimulation array 200. That is, the machine learning model is configured to receive, as an input, measured movement data (e.g., from the sensors 202) or measured motor intent data (e.g., from the electrodes 201), and output the actuation instruction corresponding to the likely motion characterized by the measured data.

In a second stage of training, the model training engine 227 can use user-specific data collected by the sensors 202 or the electrodes 201 measuring movement by the user wearing the wearable stimulation array 200. The model training engine 227 creates a second training set based on previously determined actuation instructions (e.g., by the trained general movement model 228) and the data representative of measured movement collected from the user of wearable stimulation array 200 (i.e., user-specific data). The determined actuation instructions, depending on the success of the corresponding applied actuation (e.g., as indicated by user feedback), may serve as labels for the user-specific data. If a previously determined actuation instruction resulted in stimulated movement that was effective or comfortable, the model training engine 227 may create the second training set that includes user-specific data labeled with the determined actuation instruction. The model training engine 227 then re-trains the machine learning model using the second training set such that the machine learning model is customized to the user's motions. For example, the model training engine 227 may re-train the general movement model 228 such that the re-trained model is the user-specific movement model 229.

To create a training set, the model training engine 227 may determine one or more feature vectors associated with measured movement data (e.g., a combination of kinetic signals from different muscles and the timing of their firing during the measured movement). For example, the model training engine 227 may determine a feature vector characterizing muscle firing events associated with a certain degree of knee flexion and a toe off event during a gait cycle. In some embodiments, the model training engine 227 may receive calibration data (e.g., from the calibration module 225) associated with calibration performed prior to stimulating movement. The model training engine 227 may use the calibration data in creating the training set such that the trained machine-learned model is further customized to the user's motions.

The general movement model 228 is configured to enable, for each of various movements, a corresponding actuation. For example, for each movement in a gait cycle, the general movement model 228 can determine a corresponding electrical signal to drive from a first electrode or first set of electrodes of the electrodes 201 to a second electrode or second set of electrodes of the electrodes 201. The general movement model 228 receives, as input, data representing the movement measured by the wearable stimulation array 200 and outputs an actuation instruction to assist with the movement that is likely represented in the received data. The data representing the measured movement may include EMG data, IMU data, foot plantar pressure signals, a level of fatigue of the measured movement, a context in which the stimulated movement is to occur, any suitable activity data, or combination thereof. In one example, a set of the electrodes of the electrodes 201 are configured to sense EMG signals at the shank (e.g., sensed when the user is intended to perform an ankle dorsiflexion), the EMG signals are input into the general movement model 228, and the general movement model 228 outputs an actuation instruction to stimulate an ankle dorsiflexion. The general movement model 228 is trained by the model training engine 227 using movement data, or any other suitable data representing measured movement, collected across a neurotypical population performing a variety of general movements. The general movements may include walking, standing (i.e., from a sitting position), sitting, ascending or descending steps, grasping, any suitable movement used in day-to-day activity, or a combination thereof.

The user-specific model 229 is trained by the model training engine 227 using movement data, or any other suitable data representing measured movement, collected from the sensors 202 or the electrodes 201. The model 229 may be obtained by re-training the general movement model 228. Because the model 229 may be trained on user-specific movement data, the model 229 enables the mobility augmentation system 220 to be personalized to the user and improve its accuracy in identifying actuation instructions that the user finds satisfactory (e.g., effective or comfortable). The user-specific model 229 may, similar to the general movement model 228, be configured to enable, for each of various movements, a corresponding actuation instruction.

The model training engine 227 may additionally enhance the effectiveness of the array 201 in identifying and responding to hyperexcitability events. For example, the training set used to train the user-specific movement model 229 may include data indicating the efficacy of various intervention signals applied by the electrodes 201 in response to detected hyperexcitability events. The training data may include, in some embodiments, EMG signal data collected during various instances of muscle hyperexcitability, corresponding intervention signal parameters, data indicating whether the applied intervention signals successfully reduced hyperexcitability by at least a threshold amount, and intervention signal parameters for any subsequently applied intervention signals (e.g., applied after either successful or unsuccessful intervention signals).

The trained user-specific movement model 229 may distinguish between typical EMG patterns for the user from those indicative of hyperexcitability events and more efficiently calibrate intervention signal parameters based on successful intervention signals previously applied in response to hyperexcitability events. Additionally, the user-specific movement model 229 may be periodically retrained as additional training data associated with hyperexcitability events and associated intervention signals is generated. Moreover, in some embodiments, the user-specific movement model 229 may predict future hyperexcitability events based on initial muscle signal data measured by the electrodes 201.

The general movement model 228 and user-specific movement model 229 may be machine learning models. Machine learning models of the mobility augmentation system 220 may use various machine learning techniques such as linear support vector machine (linear SVM), boosting for other algorithms (e.g., AdaBoost), neural networks, logistic regression, naïve Bayes, memory-based learning, random forests, bagged trees, decision trees, boosted trees, boosted stumps, a supervised or unsupervised learning algorithm, or any suitable combination thereof. The machine learning models may have access to a broader set of features on which to train. The models may use physiological simulation as a component for determining an actuation instruction.

Alternatively, the models described herein may be a statistical model generated based on previously measured movement data and corresponding actuation applied, the statistical model configured to determine an actuation that is most likely to correspond to measured movement data. The models described herein may also be a rules-based decision model that determines an optimal actuation based on a test of various rules or conditions such as whether the measured movement data deviates from target data by over a threshold, whether the user has been wearing the array for longer than a predetermined period of time, or any other suitable test for determining conditions under which a particular actuation should be applied.

Although the mobility augmentation system 220 is depicted as being a component of the wearable stimulation array 200, the remote mobility augmentation system 120 may provide the same or similar functionality such that the processing burden is shifted from the MCU 204 to processors local to the remote server hosting the remote mobility augmentation system 120. The data captured by the sensors 202 or EMG signals captured electrodes of the electrodes 201 may be communicated via the communications circuitry 206 to the remote mobility augmentation system 120. For example, kinetic movement data measured by the IMU sensors of the sensors 202 are stored in a Secure Digital (SD) memory card at the wearable stimulation array 200, the mobility augmentation system 220 uploads data from the SD card to a remote database (e.g., the database 130), and the remote mobility augmentation system 120 accesses the database 130 to calibrate a movement model that is accessed over the network 160 by the wearable stimulation array 200. The remote mobility augmentation system 120 may be hosted on a computing device such as a smartphone or a tablet, where the computing device can be communicatively coupled to the wearable stimulation array 200 via a communication network (e.g., the network 150).

The power source 205 provides electrical power for the wearable stimulation array 200 to operate. The power source 205 may be a mobile power source such as a battery or a fixed power source such as an outlet connection to power. The power source 205 may provide power for actuation that includes electrical stimulation via the electrodes 202. For example, the actuation coordination module 224 may activate or deactivate an electrical connection between the power source 205 and the electrodes 201 to control electrical stimulation. The power source 205 may provide power for actuation that includes mechanical stimulation via, although not depicted in FIG. 2, a vibrating motor in the wearable stimulation array 200. For example, the actuation coordination module 224 may activate or deactivate an electrical connection between the power source 205 and the vibrating motor to provide haptic or mechanical actuation.

The communications circuitry 206 enables the wearable stimulation array 200 to communicate over a network (e.g., the network 160). The communications circuitry 206 may be configured to establish a connection between the wearable stimulation array 200 and the Internet using one or more of a Wi-Fi, cellular, local area network (LAN) interface, or any suitable interface for wireless communication. The communications circuitry 206 may be configured to transmit and receive data from communications circuitry of other devices (e.g., other wearable stimulation arrays or a user device). In some embodiments, the communications circuitry 206 may also enable wired communication through various mediums such as fiber-optic, USB, serial, coaxial, or any suitable cable for wired networking.

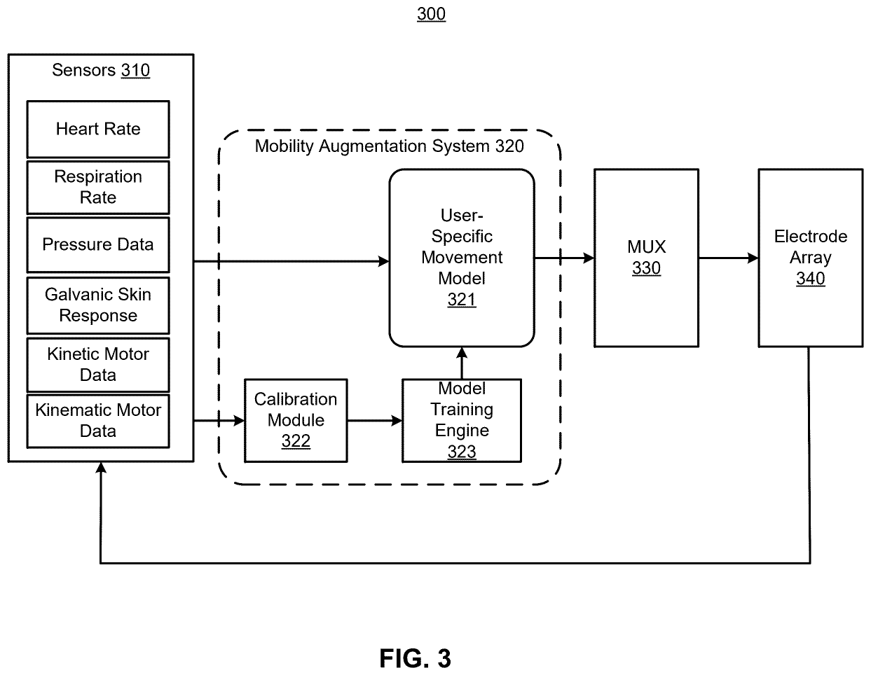

FIG. 3 is a block diagram of a feedback loop 300 for optimizing stimulation by the wearable stimulation array, in accordance with at least one embodiment. The feedback loop 300 is a closed-loop system that minimizes differences between a movement stimulated by a wearable stimulation array and a target movement (e.g., neurotypical movement). The mobility augmentation system 220 may perform the feedback loop 300. The feedback loop 300 includes sensors 310, a mobility augmentation system 320, a MUX 330, and an electrode array 340. The mobility augmentation system 320 executes a user-specific movement model 321, a calibration module 322, and a model training engine 323. The feedback loop 300 may have alternative configurations than shown in FIG. 3, including for example different, fewer, or additional components.

The optimization of stimulated movement begins with an initial application of actuation. The sensors 310 measure movement data such as a user's heart rate, respiration rate, pressure data (e.g., using pressure beds at the user's feet), galvanic skin response, kinetic movement data, kinematic movement data, or any combination thereof. The sensors 310 function similarly to the sensors 202. The sensors 310 provide the movement data to the mobility augmentation system 320, which determines an actuation instruction to apply using the user-specific movement model 321. The system 320 may be similar to the mobility augmentation system 220 described in FIG. 2 (e.g., the model 321 may function similarly to the user-specific movement model 229). The determined actuation instructions can specify a combination of electrodes to activate. The mobility augmentation system 320 uses the MUX 330 to select the specified combination (i.e., by outputting selection signal values corresponding to the specified combination). The MUX 330 functions similarly to the MUX 203. The MUX 330 enables the specified combination of electrodes in the electrode array 340 to provide electrical stimulation to the user.

The feedback needed for optimization is obtained when the sensors 310 measure the movement data representing the stimulated movement. This measurement is depicted in FIG. 3 by the arrow from the electrode array 340 to the sensors 310. Examples of feedback indicating that the level of approval of the stimulated movement is low includes measures of the body's routine functions (e.g., heart rate, respiration rate, galvanic skin response) that are outside of a normal range of values for the user (e.g., according to the user's age or height). Other examples of feedback indicating that the level of approval of the stimulated movement is low includes a comparison of the measured kinetic movement data, kinematic movement data, or pressure data against a respective set of target data that deviates beyond a predetermined threshold. For example, the level of approval of the stimulated movement may be low when the measured kinetic signals of a dorsiflexion deviate from signals of a target dorsiflexion (e.g., a dorsiflexion performed by the user during calibration or a neurotypical dorsiflexion) by ±25% of the target dorsiflexion's amplitude.

Examples of feedback indicating that the level of approval of the stimulated movement is high includes measures of the body's routine functions that are within of a normal range of values for the user. Other examples of feedback indicating that the level of approval of the stimulated movement is high includes a comparison of the measured kinetic movement data, kinematic movement data, or pressure data against a respective set of target data that meets or falls within a predetermined threshold. For example, the level of approval of the stimulated movement may be high when the amplitude of measured pressure signals from a heel strike fall within ±10% of the target heel strike's amplitude.

The feedback is used to retrain the user-specific movement model 321. The measured movement data from the sensors 310 is provided to the calibration module 322. The calibration module 322 may score the applied actuation instruction based on the level of approval of the stimulated movement, which may be determined by the mobility augmentation system 320. The level of approval may be proportional to an amount by which the stimulated movement deviates from a target movement, and the score may be proportional to the level of approval. Using the score, the model training engine 323 may create a training set of labeled data. Movement data can be labeled with an actuation instruction depending on the determined score. For example, if the score of the stimulated movement is low due to low level of approval, the model training engine 323 may create a negative sample using the applied actuation as a label to the measured movement that resulted in the unsatisfactory actuation to be applied. In another example, if the score of the stimulated movement is high due to a high level of approval, the model training engine 323 may similarly create a positive sample. The model training engine 323 can use the positive and negative samples to re-train the user-specific movement model 321 to refine the applied stimulation based on the user's body and behavior.

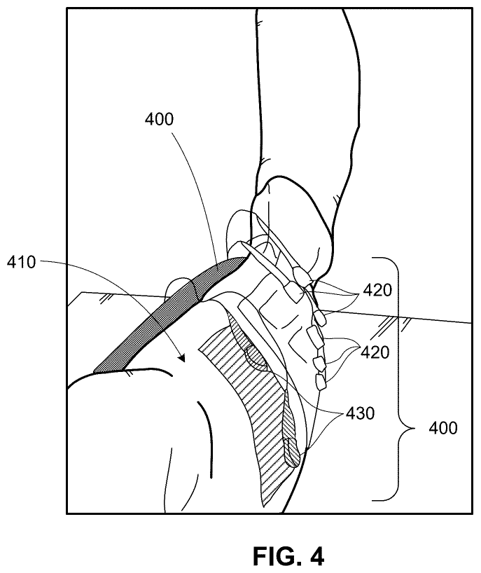

FIG. 4 depicts electrodes of a wearable stimulation array 400 in contact with a user's shank 410, in accordance with at least one embodiment. The wearable stimulation array 400 is depicted without integration into an article of clothing such as a legging or sock. The wearable stimulation array 400 includes components of the wearable stimulation array 200 such as electrodes 420 and 430. The electrodes 420 may be configured as anodes and the electrodes 430 may be configured as cathodes. The wearable stimulation array 400 depicted is an 8-electrode array having two cathodes and six anodes.

In some embodiments, a first set of the electrodes of the wearable stimulation array 400 may be configured to be electrodes for an EMG sensor of the array 400. For example, a MUX of the array 400 may select two electrodes of the 8-electrode array to be coupled to the EMG sensor. The movement data (e.g., EMG signals) sensed by the two electrodes may be applied to a movement model executed on the MCU of the array 400. The accessed movement model may output an actuation instruction determined as most optimal to stimulate the movement represented in the EMG signals. The determined actuation instruction may specify how the remaining six electrodes of the 8-electrode array may be configured (e.g., anode or cathode) and one or more electrical signals to be applied through the electrodes. The determined actuation instruction may alternatively specify that the two electrodes used for the EMG sensor be reconfigured to apply the actuation (i.e., the electrodes' roles are reconfigured from being EMG electrodes to either a cathode or anode for the electrical stimulation) to the shank 410. Disconnecting the electrodes from the EMG sensor and reconfiguring the disconnected electrodes for use in electrical stimulation may help EMG measurements avoid or reduce interference from the electrical stimulation (e.g., from the electrode MUX used to apply the stimulation).

### Graphical Depictions

FIG. 5 is a graphical depiction 500 of EMG measurements captured by electrodes 201 of the wearable stimulation array 200 before and after application of an intervention signal, in accordance with at least one embodiment. The displayed graph 500 includes representations of measurements in the amplitude domain captured during a first measurement mode 505, application of an intervention signal 510 during an intervention mode 515, and subsequent measurements captured during a second measurement mode 520. As discussed above, the same set of electrodes 201 are configured to operate in both modes such that the electrodes 201 measure EMG signals associated with one or more muscles of the user and apply an intervention signal responsive to detection of hyperexcitability of the one or more muscles.

In one embodiment, hyperexcitability is detected responsive to mean or median EMG signal amplitude (e.g., as measured in microvolts) exceeding a hyperexcitability threshold 525. For example, measurements captured by the electrodes 201 during the first measurement mode 505 indicate that one or more muscles of the user are in a hyperexcited state with mean EMG signal amplitude readings above the threshold 525.

As discussed above, responsive to the actuation coordination module 224 detecting the occurrence of a hyperexcitability event, the actuation coordination module 224 configures the electrodes 201 to operate in the intervention mode 515 such that the set of electrodes 201 apply the intervention signal 510 to the one or more muscles. As illustrated, during application of the intervention signal 510 in the intervention mode 515, the electrodes 201 do not measure EMG signals but are reconfigured to operate in the second measurement mode 520 after the intervention signal 510 is applied.

In the displayed embodiment, measurements captured during the second measurement mode 520 indicate a reduction of mean and median EMG signal measurements from the one or more muscles signaling a reduction of muscle hyperexcitability after application of the intervention signal 510. As illustrated, mean and median measured voltage during the second measurement mode 520 is below the hyperexcitability threshold 525 and is at or above a lower threshold 530 indicative of a normal excitability state. Responsive to detecting that the intervention signal was successful (i.e., reduced the muscle hyperexcitability by at least a threshold amount), the electrodes 201 continue to operate in the measurement mode until a subsequent hyperexcitability event (e.g., of the same or different muscle(s)) is detected.

While the displayed embodiment illustrates a reduction of muscle hyperexcitability by at least a threshold amount (i.e., such that the amplitude of the EMG signal readings falls below the threshold), in embodiments in which the intervention signal 510 did not reduce muscle hyperexcitability by at least the threshold amount, the calibration module 225 selects a second intervention signal (not shown) based at least in part on the second EMG signal data. For example, the calibration module 225 could select the second intervention signal by shortening or lengthening the intervention window during which the intervention signal 510 was applied or increase or decrease the frequency or amplitude of the intervention signal 510.

FIG. 6 is a graphical depiction of interleaved afferent and efferent signals applied by a set of electrodes 201 of the wearable stimulation array 200, in accordance with at least one embodiment. The displayed graph 600 includes representations of afferent signals 605 and efferent signals 610 applied over a same time period T₁-T₃ by the set of electrodes 201. As discussed above, in some embodiments, the afferent signals 605 are an initial set of low-amplitude and high-frequency afferent signals selected by the initialization module 223 upon initialization of the wearable stimulation array 200 to establish a neurological baseline of afferent feedback and prime the muscles for generating a response when a movement-inducing efferent signal is applied. Alternatively, the afferent signals 605 are a set of afferent signals selected in response to the actuation coordination module 224 detecting, using the set of electrodes 201, a measure of spasticity of one or more muscles. As illustrated in FIG. 6, only the set of afferent signals 605 are applied during an initial portion T₁ of the displayed time period.

The efferent signals 610 are selected and applied during the period T₂ in response to detection of an intended movement of the user of the wearable stimulation array 200. As illustrated in FIG. 6, while the afferent signals 605 have a consistent amplitude over the illustrated time period T₁-T₃, the amplitude of the applied efferent signals 610 increases, plateaus (e.g., at a maximum amplitude for the signal being applied), and subsequently decreases over the application period. The efferent signals 610 are also applied at a lower frequency than the afferent signals 610. The displayed configuration of efferent signals 610 illustrates one example of a set of functional motor pulses intended to stimulate an intended movement, such as pronation of the foot or a knee extension).

During the period T₂, the electrodes 201 apply both the afferent signals 605 and the efferent signals 610. As discussed above, while the afferent signals 605 and efferent signals 610 are applied over the same time period T₂, the signals 605 and 610 are interleaved. Specifically, the actuation coordination module 224 monitors the frequency and timing of the signals 605 and 610 to detect signal overlap or application of an afferent signal and an efferent signal within a threshold time period of each other. If the monitored signal frequencies indicate that an upcoming afferent signal and an upcoming efferent signal will overlap or be applied within a threshold time period of each other, the actuation coordination module 224 instructs the electrodes 201 to delay application of the upcoming afferent signal or the upcoming efferent signal or to skip the upcoming afferent signal or efferent signal. As illustrated in FIG. 6, after the efferent signals 610 are applied during the time T₂ to facilitate the intended movement, the electrodes 201 continue to apply the low-amplitude, high-frequency afferent signals 605 during the time T₃.

### Processes for Alternating Electrodes Between Measure and Intervention Modes to Address Hyperexcitability

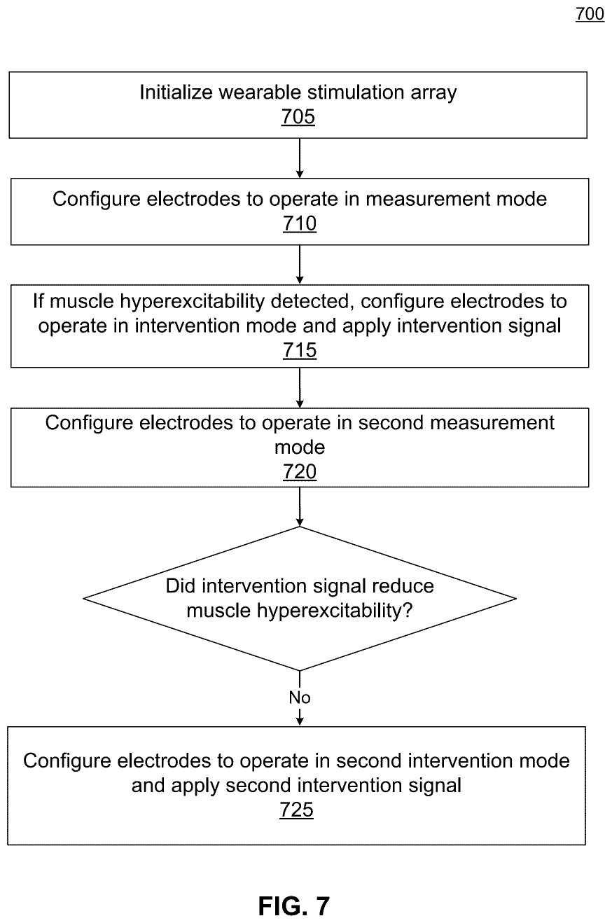

FIG. 7 is a flowchart illustrating a process 700 for alternating electrodes between measurement and intervention modes to address hyperexcitability, in accordance with at least one embodiment. The steps of FIG. 7 are illustrated from the perspective of the mobility augmentation system 200 performing the method 700. However, some or all of the steps may be performed by other entities and/or components. In addition, some embodiments may perform the steps in parallel, perform the steps in different orders, or perform different steps.

The method 700 begins with an initialization module 223 of the mobility augmentation system 220 initializing 705 the wearable stimulation array 200. The array 200 may be initialized responsive to the initialization module 223 detecting that the user has resumed wearing the array 200 based on, for example, sensor data, such as data from a heart rate sensor or a remote sensor such as a camera communicatively coupled to the mobility augmentation system 220.

Upon initialization, the initialization module 223 configures 710 the electrodes 201 of the array 200 to operate in a first measurement mode such that the electrodes 201 measure electromyography (EMG) data from one or more muscles of the user. As discussed above, default configuration of the array 200 in a measurement mode (also referred to as a “first measurement model”) ensures that EMG signal measurements are captured upon device initialization.

The actuation coordination module 224 monitors the measured EMG data reported by the electrodes 201 to identify anomalous activity data indicative of hyperexcitability of one or more muscles. For example, as discussed above, the actuation coordination module 224 compares the EMG signal data collected during the first measurement mode to at least one hyperexcitability threshold, and responsive to the EMG signal data exceeding the at least one hyperexcitability threshold (i.e., indicating muscle hyperexcitability), the actuation coordination module 224 configures 715 the electrodes 201 to operate in an intervention mode (also referred to as a “first intervention mode”) and apply an intervention signal to reduce or eliminate the identified muscle hyperexcitability.

After the electrodes 201 apply the intervention signal, the actuation coordination module 224 configures 720 the electrodes 201 to operate in a second measurement mode such that the electrodes 201 measure EMG data (also referred to as “second EMG data”) from the one or more muscles after the intervention signal is applied. The calibration module 225 evaluates the second measured EMG data to determine whether the intervention signal successfully reduced the muscle hyperexcitability by at least a threshold amount. To do so, the calibration module 225 compares the EMG data measured during the first measurement mode with the second EMG data and determines whether the amplitude, frequency, duration, or other relevant EMG parameters have decreased by more than a threshold amount. If the comparison indicates that the intervention signal was successful (i.e., muscle hyperexcitability was reduced by more than a threshold amount), the electrodes 201 of the array 200 continue to operate in the measurement mode until a subsequent hyperexcitability event is detected. Conversely, if the comparison indicates that the intervention signal did not reduce the muscle hyperexcitability by at least the threshold amount, the actuation coordination module 224 configures 725 the electrodes 201 to operate in a second intervention mode and apply a second intervention signal based on the second EMG data. The calibration module 225 may select the second intervention signal by, for example, adjusting one or more parameters of the first intervention signal, such as the amplitude, pulse frequency, duration of the intervention signal, etc.

The actuation coordination module 224 and calibration module 225 may continue to evaluate EMG data during measurement modes and adjust and apply intervention signals during intervention modes until the muscle hyperexcitability is reduced by at least a threshold amount. In this way, the mobility augmentation system 220 operates a feedback loop for optimizing intervention signals applied in response to detected hyperexcitability events.

### Process for Interleaving Efferent and Afferent Signals

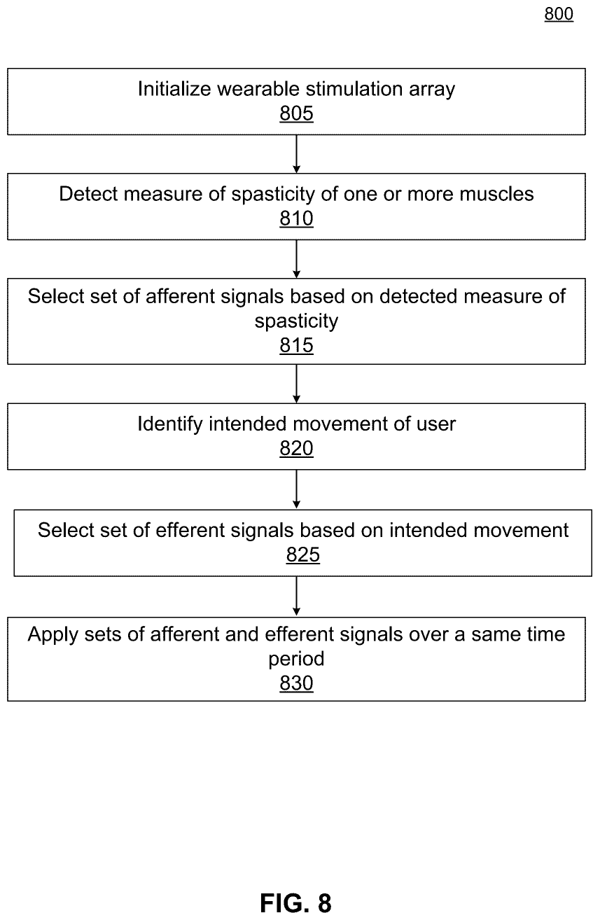

FIG. 8 is flowchart illustrating a process 800 for interleaving efferent and afferent signals, in accordance with at least one embodiment. In some embodiments, the mobility augmentation system 220 performs operations of the process 800 in parallel or in different orders, or may perform different steps.

The method 800 begins with an initialization module 223 of the mobility augmentation system 220 initializing 805 the wearable stimulation array 200. The array 200 may be initialized responsive to the initialization module 223 detecting that the user has resumed wearing the array 200 based on, for example, sensor data, such as data from a heart rate sensor or a remote sensor such as a camera communicatively coupled to the mobility augmentation system 220.

The actuation coordination module 224 detects 810 a measure of spasticity of one or more muscles of the user based on, for example, EMG data measured by the electrodes 201 indicating a level of electrical activity higher than a threshold or irregular or high-frequency bursts of electrical activity indicative of involuntary muscle contractions.

The calibration module 225 selects 815 a set of afferent signals based on the detected measure of spasticity. As discussed above, an initial set of afferent signals may be applied upon initialization of the array 200, e.g., such that an initial baseline of afferent signals allow the user's nervous system to adapt to the presence of artificial stimulations and prime the muscles for generating a response when functional motor signals are applied. In one embodiment, to select the set of afferent signals based on the detected spasticity, the calibration module 225 adjusts one or more parameters of the initial set of afferent signals, such as by increasing or decreasing the amplitude, frequency, or pattern of the afferent signals.

The actuation coordination module 224 identifies 820 an intended movement of the user, e.g., based on EMG data, IMU data, foot plantar pressure signals, a context in which the movement occurs, or a combination thereof and selects 825 a set of efferent signals based on the intended movement. As discussed above, the efferent signals are intended facilitate the identified movement and may be selected based on a predefined mapping associating specific movements with corresponding sets of efferent signals or output from a machine learning model, such as the general movement model 228 or the user-specific movement model 229. In various embodiments, selected sets of efferent signals may be fine-tuned based on user feedback or other factors, such as muscle tone, degree of spasticity, fatigue level, and the like.

Finally, the electrodes 201 of the wearable stimulation array 200 apply 830 the set of afferent signals and the set of efferent signals over the same period of time such that the set of afferent signals and the set of efferent signals are interleaved. In one embodiment, the actuation coordination module 224 coordinates the interleaved application of the signals by instructing the electrodes 201 to sequentially apply the signals to avoid overlap (e.g., to avoid a high amplitude pulse created by simultaneous application of an afferent signal and an efferent signal). To do so, the actuation coordination module 224 monitors the frequency and timing of the selected sets of afferent and efferent signals and instructs the electrodes 201 to delay or skip application of an afferent signal or an efferent signal responsive to detecting that an upcoming afferent signal and an upcoming efferent signal will overlap or occur within a threshold time period of each other. Accordingly, tone reducing afferent pulses and functional motor activation efferent pulses may be effectively applied over the same time period without interfering with each other.

### Additional Considerations

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm may be a sequence of operations leading to a desired result. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

It should be borne in mind, however, that all of these and similar terms are to be associated with the appropriate physical quantities and are merely convenient labels applied to these quantities. Unless specifically stated otherwise as apparent from the present disclosure, it is appreciated that throughout the description, certain terms refer to the action and processes of a computer system, or similar electronic computing device, that manipulates and transforms data represented as physical (electronic) quantities within the computer system's registers and memories into other data similarly represented as physical quantities within the computer system memories or registers or other such information storage devices.

The present disclosure also relates to an apparatus for performing the operations herein. This apparatus may be specially constructed for the intended purposes, or it may include a computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMS, EEPROMs, magnetic or optical cards, or any type of media suitable for storing electronic instructions, each coupled to a computer system bus.

The algorithms and displays presented herein are not inherently related to any particular computer or other apparatus. Various other systems may be used with programs in accordance with the teachings herein, or it may prove convenient to construct a more specialized apparatus to perform the method. In addition, the present disclosure is not described with reference to any particular programming language. It will be appreciated that a variety of programming languages may be used to implement the teachings of the disclosure as described herein.

The present disclosure may be provided as a computer program product, or software, that may include a machine-readable medium having stored thereon instructions, which may be used to program a computer system (or other electronic devices) to perform a process according to the present disclosure. A machine-readable medium includes any mechanism for storing information in a form readable by a machine (e.g., a computer). For example, a machine-readable (e.g., computer-readable) medium includes a machine (e.g., a computer) readable storage medium such as a read only memory (“ROM”), random access memory (“RAM”), magnetic disk storage media, optical storage media, flash memory devices, etc.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment. Where values are described as “approximate” or “substantially” (or their derivatives), such values should be construed as accurate +/−10% unless another meaning is apparent from the context. From example, “approximately ten” should be understood to mean “in a range from nine to eleven.”

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present). In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A method comprising:
initializing a wearable stimulation array comprising a plurality of electrodes;
configuring a set of the electrodes to operate in a measurement mode such that the set of electrodes, when configured in the measurement mode, can measure an electromyography (EMG) signal from one or more muscles;
in response to determining that the measured EMG signal is representative of muscle hyperexcitability, configuring the set of electrodes to operate in an intervention mode such that the set of electrodes applies an intervention signal to the one or more muscles;
configuring the set of electrodes to operate in a measurement mode to measure a second EMG signal from the one or more muscles; and
in response to determining that the intervention signal did not reduce the hyperexcitability of the one or more muscles by at least a threshold amount, configuring the set of electrodes to operate in the intervention mode such that the set of electrodes applies a second intervention signal to the one or more muscles, the second intervention signal based at least in part on the second EMG signal.

2. The method of claim 1, where the wearable stimulation array determines that the measured EMG signal is representative of muscle hyperexcitability based on a comparison between one or more parameters of the measured EMG signal and at least one hyperexcitability threshold.

3. The method of claim 1, wherein the applied intervention signal is selected based on one or more of a type of hyperexcitability event detected, a severity of the hyperexcitability event, and the one or more muscles associated with the hyperexcitability event.

4. The method of claim 1, wherein the second intervention signal is different from the intervention signal.

5. The method of claim 1, wherein the second intervention signal is determined by adjusting at least one signal parameter of the intervention signal.

6. The method of claim 1, wherein the wearable stimulation array determines that the intervention signal did not reduce the hyperexcitability by at least a threshold amount based on a comparison between one or more parameters of the EMG signal and one or more parameters of the second EMG signal.

7. The method of claim 1, further comprising:
configuring the set of electrodes to operate in a measurement mode to measure a third EMG signal from the one or more muscles;
responsive to determining that the second intervention signal reduced the hyperexcitability of the one or more muscles by at least a threshold amount, storing an association between the muscle hyperexcitability of the one or more muscles and the second intervention signal in a user profile; and
responsive to identifying a reoccurrence of the muscle hyperexcitability based on subsequent EMG signal data from the one or more muscles, configuring the set of electrodes to operate in the intervention mode such that the set of electrodes applies the second intervention signal to the one or more muscles.

8. The method of claim 1, further comprising:
configuring the set of electrodes to operate in a measurement mode to measure a third EMG signal from the one or more muscles;
responsive to determining that the second intervention signal reduced the hyperexcitability of the one or more muscles by at least a threshold amount, storing an association between the muscle hyperexcitability of the one or more muscles and the second intervention signal in a user profile; and
responsive to identifying a reoccurrence of the muscle hyperexcitability based on subsequent EMG signal data from the one or more muscles, configuring the set of electrodes to operate in the intervention mode such that the set of electrodes applies a third intervention signal to the one or more muscles, the third intervention signal different from the intervention signal and the second intervention signal.

9. The method of claim 1, further comprising:
accessing a machine-learning model configured to, for each of a plurality of muscle hyperexcitability events, identify a corresponding intervention signal intended to reduce the hyperexcitability of one or more muscles;
creating a training set including data indicating an efficacy of intervention signals applied by the set of electrodes in response to detected hyperexcitability events; and
training the machine-learning model using the training set.

10. The method of claim 9, further comprising: responsive to identifying a reoccurrence of the muscle hyperexcitability based on subsequent EMG signal data from the one or more muscles, applying the machine-learning model to identify an intervention signal likely to reduce the hyperexcitability of the one or more muscles.

11. A wearable stimulation array comprising a non-transitory computer-readable storage medium storing instructions for execution and a hardware processor configured to execute the instructions, the instructions, when executed, cause the hardware processor to perform steps comprising:
initializing a wearable stimulation array comprising a plurality of electrodes;
configuring a set of the electrodes to operate in a measurement mode such that the set of electrodes, when configured in the measurement mode, can measure an electromyography (EMG) signal from one or more muscles;
in response to determining that the measured EMG signal is representative of muscle hyperexcitability, configuring the set of electrodes to operate in an intervention mode such that the set of electrodes applies an intervention signal to the one or more muscles;
configuring the set of electrodes to operate in a measurement mode to measure a second EMG signal from the one or more muscles; and
in response to determining that the intervention signal did not reduce the hyperexcitability of the one or more muscles by at least a threshold amount, configuring the set of electrodes to operate in the intervention mode such that the set of electrodes applies a second intervention signal to the one or more muscles, the second intervention signal based at least in part on the second EMG signal.

12. The wearable stimulation array of claim 11, where the wearable stimulation array determines that the measured EMG signal is representative of muscle hyperexcitability based on a comparison between one or more parameters of the measured EMG signal and at least one hyperexcitability threshold.

13. The wearable stimulation array of claim 11, wherein the applied intervention signal is selected based on one or more of a type of hyperexcitability event detected, a severity of the hyperexcitability event, and the one or more muscles associated with the hyperexcitability event.

14. The wearable stimulation array of claim 11, wherein the second intervention signal is different from the intervention signal.

15. The wearable stimulation array of claim 11, wherein the second intervention signal is determined by adjusting at least one signal parameter of the intervention signal.

16. The wearable stimulation array of claim 11, wherein the wearable stimulation array determines that the intervention signal did not reduce the hyperexcitability by at least a threshold amount based on a comparison between one or more parameters of the EMG signal and one or more parameters of the second EMG signal.

17. The wearable stimulation array of claim 11, wherein the instructions further cause the hardware processor to:
configure the set of electrodes to operate in a measurement mode to measure a third EMG signal from the one or more muscles;
responsive to determining that the second intervention signal reduced the hyperexcitability of the one or more muscles by at least a threshold amount, store an association between the muscle hyperexcitability of the one or more muscles and the second intervention signal in a user profile; and
responsive to identifying a reoccurrence of the muscle hyperexcitability based on subsequent EMG signal data from the one or more muscles, configure the set of electrodes to operate in the intervention mode such that the set of electrodes applies the second intervention signal to the one or more muscles.

18. The wearable stimulation array of claim 11, wherein the instructions further cause the hardware processor to:
configure the set of electrodes to operate in a measurement mode to measure a third EMG signal from the one or more muscles;
responsive to determining that the second intervention signal reduced the hyperexcitability of the one or more muscles by at least a threshold amount, store an association between the muscle hyperexcitability of the one or more muscles and the second intervention signal in a user profile; and
responsive to identifying a reoccurrence of the muscle hyperexcitability based on subsequent EMG signal data from the one or more muscles, configure the set of electrodes to operate in the intervention mode such that the set of electrodes applies a third intervention signal to the one or more muscles, the third intervention signal different from the intervention signal and the second intervention signal.

19. The wearable stimulation array of claim 11, wherein the instructions further cause the hardware processor to:
access a machine-learning model configured to, for each of a plurality of muscle hyperexcitability events, identify a corresponding intervention signal intended to reduce the hyperexcitability of one or more muscles;
create a training set including data indicating an efficacy of intervention signals applied by the set of electrodes in response to detected hyperexcitability events; and
train the machine-learning model using the training set.

20. A non-transitory computer readable storage medium storing executable instructions that, when executed by one or more processors, cause the one or more processors to perform steps comprising:
initializing a wearable stimulation array comprising a plurality of electrodes;
configuring a set of the electrodes to operate in a measurement mode such that the set of electrodes, when configured in the measurement mode, can measure an electromyography (EMG) signal from one or more muscles;
in response to determining that the measured EMG signal is representative of muscle hyperexcitability, configuring the set of electrodes to operate in an intervention mode such that the set of electrodes applies an intervention signal to the one or more muscles;
configuring the set of electrodes to operate in a measurement mode to measure a second EMG signal from the one or more muscles; and
in response to determining that the intervention signal did not reduce the hyperexcitability of the one or more muscles by at least a threshold amount, configuring the set of electrodes to operate in the intervention mode such that the set of electrodes applies a second intervention signal to the one or more muscles, the second intervention signal based at least in part on the second EMG signal.
